(19) United States
(12) Patent Application Publication
(10) Pub. No.: US 2024/0296286 A1
(43) Pub. Date: Sep. 5, 2024

(54) METHODS AND SYSTEMS FOR PERSONAL RECIPE GENERATION

(71) Applicant: KPN INNOVATIONS LLC, LAKEWOOD, CO (US)

(72) Inventor: Kenneth Neumann, LAKEWOOD, CO (US)

(73) Assignee: KPN INNOVATIONS LLC, LAKEWOOD, CO (US)

(21) Appl. No.: 18/661,304

(22) Filed: May 10, 2024

Related U.S. Application Data
(63) Continuation-in-part of application No. 17/540,335, filed on Dec. 2, 2021, now Pat. No. 12,001,796.
Application No. 17/540,335 is a continuation of application No. 17/106,793, filed on Nov. 30, 2020, now Pat. No. 11,232,259.

(51) Int. Cl. G06F 40/284; G06F 16/9035; G06F 40/237; G06F 40/30; G16H 20/60

(52) U.S. Cl. CPC G06F 40/284; G06F 16/9035; G06F 40/237; G06F 40/30; G16H 20/60

(57) ABSTRACT

A system for informing cooking style decisions, the system includes a computing device designed and configured to receive a personal recipe relating to a user. The computing device also designed and configured to select a cooking style, including locating in a recipe database a set of cooking styles associated with the personal recipe and selecting the cooking style from the set of cooking styles as a function of the user's biological extraction. Additionally, the computing device is designed and configured to incorporate the cooking style into the personal recipe of the user.

### CROSS-REFERENCE TO RELATED APPLICATIONS

This application is a continuation-in-part of Non-provisional application Ser. No. 17/540,335 filed on Dec. 2, 2021, and entitled “METHODS AND SYSTEMS FOR PERSONAL RECIPE GENERATION,” which is a continuation of Non-provisional application Ser. No. 17/106,793 filed on Nov. 30, 2020, now U.S. Pat. No. 11,232,259, and entitled “METHODS AND SYSTEMS FOR PERSONAL RECIPE GENERATION,” both of which are incorporated herein by reference in their entirety.

### FIELD OF THE INVENTION

The present invention generally relates to the field of nourishment. In particular, the present invention is directed to methods and systems for personal recipe generation.

### BACKGROUND

Personal recipe generation can be challenging. Knowing what nutrients and quantities of nutrients individually is difficult to ascertain.

### SUMMARY OF THE DISCLOSURE

In an aspect, a system for informing cooking style decisions, the system including a computing device. The computing device is configured to identify a nutrient anomaly relating to a user. The computing device is configured to determine a target profile utilizing the nutrient anomaly. The computing device is configured to generate a personal recipe relating to the user. Generating the personal recipe comprises selecting a plurality of ingredient identifiers as a function of the target profile, wherein each ingredient identifier is associated with an ingredient impact, filtering the plurality of ingredient identifiers as a function of ingredient accessibility, and generating the personal recipe using a plurality of ingredient impacts associated with the plurality of ingredient identifiers. The computing device is configured to select a cooking style. Selecting the cooking style comprises locating, in a recipe database, a cooking style table comprising a set of cooking styles associated with the personal recipe and selecting the cooking style from the set of cooking styles as a function of the target profile. The computing device is configured to generate a recipe depiction as a function of the cooking style and the personal recipe.

In another aspect, a method for informing cooking style decisions is disclosed. The method comprises identifying, using at least a computing device, a nutrient anomaly relating to a user. The method comprises determining, using the at least a computing device, a target profile utilizing the nutrient anomaly. The method comprises generating, using the at least a computing device, a personal recipe relating to the user. Generating the personal recipe comprises selecting a plurality of ingredient identifiers as a function of the target profile, wherein each ingredient identifier is associated with an ingredient impact, filtering the plurality of ingredient identifiers as a function of ingredient accessibility, and generating the personal recipe using a plurality of ingredient impacts associated with the plurality of ingredient identifiers. The method comprises selecting a cooking style. Selecting the cooking style comprises locating, in a recipe database, a cooking style table comprising a set of cooking styles associated with the personal recipe and selecting the cooking style from the set of cooking styles as a function of the target profile. The method comprises generating a recipe depiction as a function of the cooking style and the personal recipe.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram illustrating an exemplary embodiment of a system for personal recipe generation;

FIG. 2 is a block diagram illustrating an exemplary embodiment of a user database;

FIG. 3 is a block diagram illustrating an exemplary embodiment of an ingredient database;

FIG. 4 is a block diagram illustrating an exemplary embodiment of a recipe database;

FIG. 5 is a flow diagram illustrating an exemplary embodiment of a machine learning module;

FIG. 6 is a diagrammatic representation of aspects of a user database;

FIG. 7 is a diagrammatic representation of a target profile;

FIG. 8 is a block diagram illustrating an exemplary embodiment of a filtering system;

FIG. 9 is a flow diagram illustrating an exemplary embodiment of a method of personal recipe generation; and

FIG. 10 is a block diagram of a computing system that can be used to implement any one or more of the methodologies disclosed herein and any one or more portions thereof.

The drawings are not necessarily to scale and may be illustrated by phantom lines, diagrammatic representations, and fragmentary views. In certain instances, details that are not necessary for an understanding of the embodiments or that render other details difficult to perceive may have been omitted.

### DETAILED DESCRIPTION

At a high level, aspects of the present disclosure are directed to systems and methods for a system for personal recipe generation. In an embodiment, a first ingredient identifier containing a first ingredient impact is compared to a target profile. A target profile may be generated utilizing information pertaining to a user to identify a nutrient target computed for a measured time interval. A personal recipe is then generated taking into account user specific needs, adjustments, and customization.

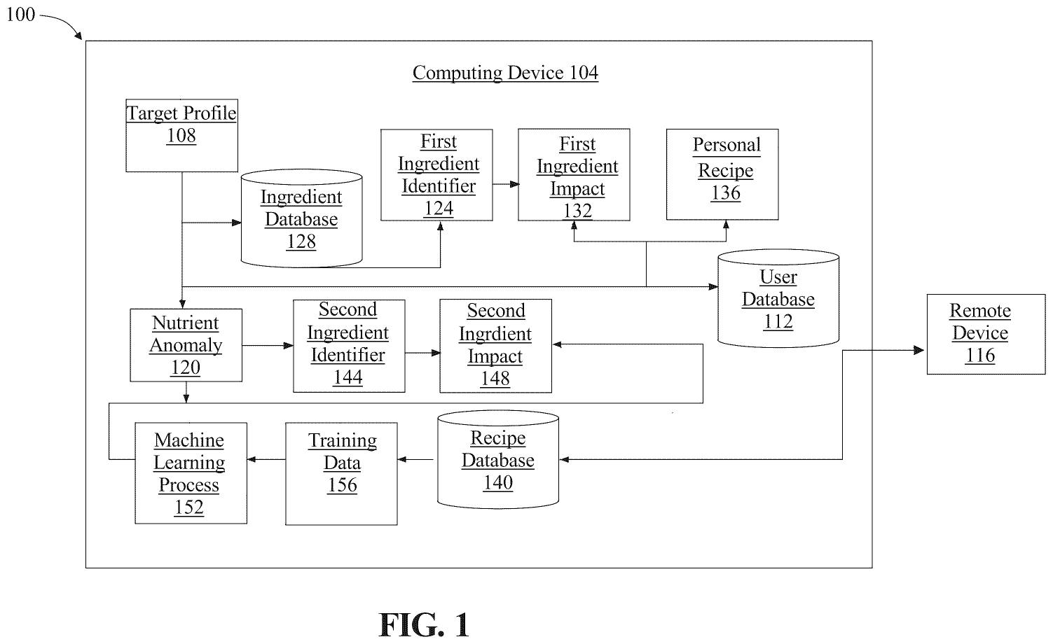

Referring now to FIG. 1, an exemplary embodiment of a system 100 for personal recipe generation is illustrated. System includes a computing device 104. Computing device 104 may include any computing device as described in this disclosure, including without limitation a microcontroller, microprocessor, digital signal processor (DSP) and/or system on a chip (SoC) as described in this disclosure. Computing device may include, be included in, and/or communicate with a mobile device such as a mobile telephone or smartphone. Computing device 104 may include a single computing device operating independently, or may include two or more computing device operating in concert, in parallel, sequentially or the like; two or more computing devices may be included together in a single computing device or in two or more computing devices. Computing device 104 may interface or communicate with one or more additional devices as described below in further detail via a network interface device. Network interface device may be utilized for connecting computing device 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Computing device 104 may include but is not limited to, for example, a computing device or cluster of computing devices in a first location and a second computing device or cluster of computing devices in a second location. Computing device 104 may include one or more computing devices dedicated to data storage, security, distribution of traffic for load balancing, and the like. Computing device 104 may distribute one or more computing tasks as described below across a plurality of computing devices of computing device, which may operate in parallel, in series, redundantly, or in any other manner used for distribution of tasks or memory between computing devices. Computing device 104 may be implemented using a “shared nothing” architecture in which data is cached at the worker, in an embodiment, this may enable scalability of system 100 and/or computing device.

With continued reference to FIG. 1, computing device 104 may be designed and/or configured to perform any method, method step, or sequence of method steps in any embodiment described in this disclosure, in any order and with any degree of repetition. For instance, computing device 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

With continued reference to FIG. 1, computing device 104 is configured to receive a target profile 108 relating to a user. A “target profile,” as used in this disclosure, is a recommended nutrient intake for a living subject, such as a human being and/or an animal. A “nutrient,” as used in this disclosure, is a substance that provides nourishment essential for growth and/or maintenance of life for a living subject. A nutrient may include for example, protein, carbohydrates, fats, lipids, vitamins, minerals, trace minerals, water, and the like. A target profile 108 may be a nutrient target computed for a measured time interval. A “nutrient target,” as used in this disclosure, is a specified target of one or more recommended nutrients that the user is recommended to consume. A nutrient target may include a measured time interval, whereby the nutrient target may contain a recommended dose and/or time limit for consumption. For instance and without limitation, a target profile 108 may contain a recommended nutrient intake for a user who is a thirty-seven-year-old pregnant female which contains nutrient recommendations such as 1000 micrograms of folate per day, and 1200 milligrams of calcium per day. In yet another non-limiting example, a target profile 108 may contain a recommended nutrient intake for a user who is a sixty-two-year-old male which contains nutrient recommendations such as twenty-five grams of fiber per day, 1 milligram of Vitamin B12 per day, and fifty-six grams of protein each day. In an embodiment, a target profile 108 may identify a nutrient target computed for a measured time interval. For example, a target profile 108 may contain a recommended nutrient target over a certain period of time such as a recommendation per hour, per day, per week, per month, and the like. In an embodiment, a measured time internal may include a custom dosing schedule, such as a recommendation for a user to consume a dose of Vitamin C three times per day. In an embodiment, a measured time internal may include a dosing schedule such as once in the morning at 6:00 am and once in the evening at 6:00 pm. In an embodiment, target profile 108 may contain information pertaining to previous meal choices and/or selections that a user may have consumed and/or ordered. Such information may be retrieved from user database 112.

With continued reference to FIG. 1, information pertaining to a target profile 108 may be contained within user database 112. User database 112 may be implemented, without limitation, as a relational database, a key-value retrieval datastore such as a NOSQL database, or any other format or structure for use as a datastore that a person skilled in the art would recognize as suitable upon review of the entirety of this disclosure. User database 112 may include information pertaining to a user and/or a user's lifestyle that may be utilized to generate a target profile 108. For instance and without limitation, user database 112 may contain information that includes a user's current eating habits, allergies, food likes, food dislikes, activity levels, medical records, medical conditions and the like, as described below in more detail. Information contained within user database 112 may be stored within computing device 104, and/or received from remote device 116. Remote device 116 may contain any additional computing device, such as a mobile device, laptop, desktop, computer, and the like. Remote device 116 may receive information pertaining to a user and store the information pertaining to the user within user database 112 utilizing any network methodology as described herein.

With continued reference to FIG. 1, computing device 104 is configured to identify a nutrient anomaly 120 relating to a user and determine a target profile 108 utilizing the nutrient anomaly 120. A “nutrient anomaly,” as used in this disclosure, is an abnormality relating to a user's current nutrient status and/or requirements. A nutrient anomaly 120 may include a nutrient deficiency and/or a nutrient excess. A “nutrient deficiency,” as used in this disclosure, is a lack or shortage of one or more nutrients. A nutrient deficiency may occur due to various behaviors and/or factors that may affect a user, such as for example inadequate intake, altered absorption, increased requirement, increased losses, and/or altered metabolism. For instance, and without limitation, a menstruating female may be deficient in iron due to an increased loss of blood during monthly menstruations. In yet another non-limiting example, an alcoholic may be deficient in Vitamin B1 due to excess consumption of alcohol which may decrease levels of B vitamins. A “nutrient excess,” as used in this disclosure, is an excess of a nutrient. A nutrient excess may occur due to various factors that may include for example improper intake, improper supplementation, malfunctioning of neuroendocrine system, toxins, drugs, and the like. For example, a nutrient excess may occur in a teenager who may consume an excess quantity of carbohydrates. A nutrient anomaly 120 may be self-reported by a user, such as a user who is a diabetic and who may self-report a nutrient anomaly 120 recommending the user to consume no more than 40 grams of carbohydrates at each meal. A nutrient anomaly 120 may be identified by computing device 104 based on information and/or medical records that may be contained within user database 112. For instance, and without limitation, information pertaining to a user and stored within user database 112 may specify that a user is currently taking a statin medication to lower the user's cholesterol levels. In such an instance, computing device 104 may detect a nutrient anomaly that specifies that the user requires an additional dose of co-enzyme q10, due to a nutrient deficiency exacerbated by statin medication intake. Computing device 104 may determine a target profile 108 using a nutrient anomaly 120. A nutrient anomaly 120 may contain a temporal element. A “temporal element,” as used in this disclosure, is any information relating to time. A temporal element may specify how long a nutrient anomaly 120 has occurred. A temporal element may specify how long a nutrient anomaly 120 may impact a target profile 108. For instance, and without limitation, a nutrient anomaly 120 such as a user with a ferritin level of 5 micrograms per liter may contain a temporal element indicating that the user will require extra intake of iron for at least six months. In yet another non-limiting example, a nutrient anomaly 120 such as a user with colitis may contain a temporal element indicating that the user will require 5 grams per day of soluble fiber for the duration of colitis.

With continued reference to FIG. 1, a first nutrient anomaly may be identified as a function of a second nutrient anomaly. This may occur for example, such as where a first nutrient deficiency and/or excess may cause a deficiency and/or excess of a second nutrient deficiency. For instance, and without limitation, a first nutrient anomaly such as a zinc deficiency may cause a subsequent deficiency of a second nutrient anomaly such as copper. This may also occur where the replenishment of a first nutrient anomaly requires the replenishment of a second nutrient anomaly. For instance, and without limitation, the replenishment of a first nutrient anomaly such as Vitamin B1 or thiamine may require the replenishment of a second nutrient anomaly such as Vitamin B2 or riboflavin. This may also occur where a first nutrient anomaly may be related to a second nutrient anomaly. For instance, and without limitation, a first nutrient anomaly such as an iron deficiency may be related to a second nutrient anomaly such as a ferritin deficiency. In yet another non-limiting example, a first nutrient anomaly such as a selenium deficiency may be related to a second nutrient anomaly such as an iodine deficiency.

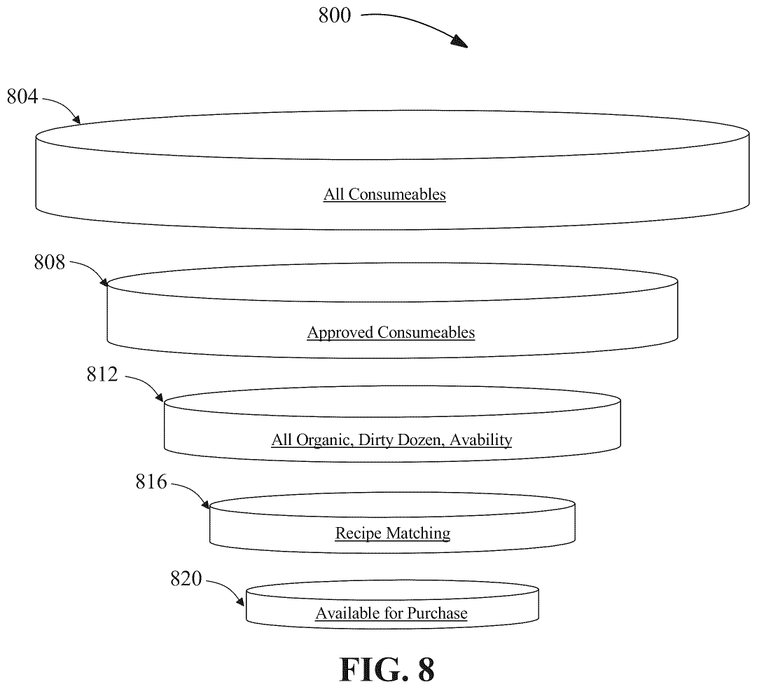

With continued reference to FIG. 1, computing device 104 is configured to locate in a lexicon of ingredients wherein the lexicon associates a plurality of ingredient identifiers to a plurality of ingredient impacts and a plurality of semantic units, a first ingredient identifier including a first ingredient impact and a first semantic unit. An “ingredient identifier,” as used in this disclosure, is a unique sequence of characters and/or numerical values, used to identify an ingredient. An “ingredient,” as used in this disclosure, is any food, beverage, meal, snack, and/or substance that may be combined to make a particular dish and/or meal. An ingredient may include, for example, a vegetable such as an artichoke or a fruit such as an apple. An ingredient may include, for example, a protein source such as poultry, eggs, nuts, fish, beans, meat, tofu, seitan, and the like. An ingredient may include a grain such as bread, cereal, rice, pasta, millet, and the like. An ingredient may include beverages such as cow's milk or non-dairy alternatives such as coconut milk or soy milk. An ingredient may include an oil such as olive oil or coconut oil. An ingredient may include a baking essential such as brown sugar or soy sauce. A “lexicon,” as used in this disclosure, is any data structure suitable for use as user database 112 as described above. A “semantic unit,” as used in this disclosure, is any textual data. A semantic unit may contain information describing a category and/or class of a first ingredient identifier. For example, a semantic unit may indicate if a first ingredient identifier belongs to a category such as a vegetable, a fruit, a spice, a protein source, and the like. For instance, and without limitation, a first ingredient identifier such as a tomato may be associated with a semantic unit such as vegetable. In yet another non-limiting example, a first ingredient identifier such as filet mignon may be associated with a semantic unit such as animal protein. Information pertaining to semantic units may be updated in real time, utilizing any network methodology as described herein. In an embodiment, the lexicon of ingredients may include all consumable items that are accessible to the user. Consumable items may include food items that can be consumed without injuring the user. Computing device 104 may filter the list of consumable items according to their availability, target profile, nutritional needs, user preference, allergies, and the like. This filtering system may be done multiple times until the desired ingredients are identified. Filtering system may be the filtering system discussed herein below in FIG. 8.

With continued reference to FIG. 1, a first ingredient identifier 124 contains a first ingredient impact 132. An “ingredient impact,” as used in this disclosure, is data including any numerical, character, and/or symbolic data, describing nutrients contained within an ingredient. In an embodiment, nutrients contained within an ingredient may be based on one or more nutritional systems created and/or implemented by governments, non-profit organizations, private institutions, companies, and the like. For example, an ingredient impact 124 may indicate that an ingredient such as broccoli contains 10 grams of carbohydrates and 4 grams of fiber in a 150-gram serving. In yet another non-limiting example, an ingredient impact 124 may indicate that an ingredient such as wild caught salmon contains 23 grams of protein, 4 grams of fat, 85 milligrams of cholesterol, and 1200 milligrams of omega-3 fatty acids in a 4-ounce serving. Information pertaining to ingredient identifiers and/or ingredient impacts may be contained within ingredient database 128. In an embodiment, an ingredient impact may be determined using a user's biological extraction, which may indicate how well a user may absorb a particular nutrient, metabolize a certain ingredient, excrete a vitamin or mineral and the like. A “biological extraction,” as used in this disclosure, is data indicative of a person's biological state; biological state may be evaluated with regard to one or more measures of health of a person's body, one or more systems within a person's body such as a circulatory system, a digestive system, a nervous system, or the like, one or more organs within a person's body, and/or any other subdivision of a person's body useful for diagnostic or prognostic purposes. For instance, and without limitation, a particular set of biomarkers, test results, and/or biochemical information may be recognized in a given medical field as useful for identifying various disease conditions or prognoses within a relevant field. As a non-limiting example, and without limitation, biological data describing red blood cells, such as red blood cell count, hemoglobin levels, hematocrit, mean corpuscular volume, mean corpuscular hemoglobin, and/or mean corpuscular hemoglobin concentration may be recognized as useful for identifying various nourishments such as dehydration, nutrient deficiencies, anemia, and/or blood loss. Biological extraction data may alternatively or additionally include any data used as a biological extraction as described in U.S. Nonprovisional application Ser. No. 16/502,835, filed on Jul. 3, 2019, and entitled “METHODS AND SYSTEMS FOR ACHIEVING VIBRANT CONSTITUTION BASED ON USER INPUTS,” the entirety of which is incorporated herein by reference.

With continued reference to FIG. 1, computing device 104 may locate a first ingredient identifier 124 based on a user preference. A “user preference,” as used in this disclosure, is an affinity and/or dislike for one or more ingredients. For example, a user preference may indicate that a user enjoys protein sources that include chicken, beef, and fish, and that the user dislikes protein sources that include eggs, tofu, and pork. In yet another non-limiting example, a user preference may indicate that a user does not consume animal containing products because of ethical concerns. A user preference may highlight eating patterns of a user, including how many meals per day the user typically consumes, what types of foods and ingredients the user enjoys consuming, what types of foods and ingredients the user dislikes, any allergies the user has, and/or ingredients the user may abstain from consuming due to ethical and/or religious beliefs. Information pertaining to a user preference may be stored within user database 112. Based on the information stored within user database 112, which includes details about a user's eating patterns, preferences, dislikes, allergies, and dietary restrictions due to ethical or religious beliefs, computing device 104 may be configured to actively monitor ingredient selections. If an ingredient is chosen that conflicts with the user's allergies or violates their ethical or religious dietary constraints, the device will automatically trigger a notification. This notification is designed to alert the user promptly about the potential issue, providing an opportunity to make necessary adjustments to the recipe. This feature ensures that the user's dietary needs and preferences are respected and maintained throughout the cooking process, enhancing safety and personalization of the cooking experience. In an embodiment, information pertaining to a user preference may be collected from a user using a questionnaire and/or series of questions to prompt a user for information. Such information may be collected utilizing remote device 116 and/or any network methodology as described herein. Computing device 104 may utilize a user preference to locate a first ingredient identifier 124. For example, a user preference that indicates a user dislikes shellfish, may be utilized to select a first ingredient identifier 124 that contains a different fish option, such as tuna or swordfish. In yet another non-limiting example, a user preference that indicates a user likes oranges may be utilized to select other ingredients that contain citrus fruits such as grapefruit, line, lemon, kumquat, clementine, tangelo, and the like. In an embodiment, computing device 104 may identify a first ingredient identifier 124 based on a user preference, utilizing model known as a classifier. A “classifier,” as used in this disclosure, is a process whereby computing device 104 derives from training data, a model known as a “classifier” for sorting inputs into categories or bins of data. Classification algorithms may include linear classifiers such as logistic regression, Naïve Bayes classification, Fisher's linear discriminant, k-nearest neighbors, support vector machines, quadratic classifiers, Kernel estimation, decision trees, boosted trees, random forest, neural networks, and the like. Classification may be performed as described in U.S. Nonprovisional application Ser. No. 16/887,388, filed on May 29, 2020, and entitled “METHODS, SYSTEMS, AND DEVICES FOR GENERATING A REFRESHMENT INSTRUCTION SET BASED ON INDIVIDUAL PREFERENCES,” the entirety of which is incorporated herein by reference.

With continued reference to FIG. 1, computing device 104 may select an ingredient identifier 124 based on ingredient accessibility. As used in the current disclosure, “ingredient accessibility” refers to the case with which ingredients can be obtained by a user for cooking or production purposes. This the availability of specific ingredients may directly influence the functionality and effectiveness of computing device 104 to produce a recipe. Ingredient accessibility can be affected by several factors including geographic location, seasonal variations, price, and the like. In an embodiment, computing device 104 may evaluate the availability of the one or more ingredients within a geographic radius of a user. In a non-limiting example, if a recipe calls for the use of crawfish, computing device 104 may evaluate the availability of crawfish within a 10-mile radius of the current location of the user. Computing device 104 may achieve this by searching the inventories and/or websites of stores within the radius. In some cases, certain ingredients may be readily available in urban areas but scarce in rural locations. Similarly, seasonal produce may only be accessible during particular times of the year. Computing device 104 may adjust recipes, suggest alternatives, or even influence shopping lists as function of the ingredient accessibility.

With continued reference to FIG. 1, computing device 104 may select an ingredient identifier 124 based on the proximity of ingredients to the user's location. Proximity affects availability and plays a significant role in the functionality and effectiveness of computing device 104 in generating feasible recipe suggestions. In an embodiment, computing device 104 may evaluate the physical proximity of ingredients by integrating location-based services and mapping technologies. This may involve using GPS data or user-provided location information to pinpoint the nearest grocery stores, farmers' markets, and other food vendors. The device may then query these locations for the availability of specific ingredients required by the user's selected recipe. To accomplish this, computing device 104 may access a network of retail inventory systems through APIs that provide real-time or regularly updated stock information. For instance, if a user needs fresh basil, computing device 104 may automatically query nearby stores to check if basil is in stock and inform the user of the closest location where it can be purchased. This process can be further enhanced by incorporating user preferences for certain stores or types of shopping experiences (e.g., organic markets versus conventional supermarkets). Moreover, computing device 104 might also take into account the operating hours and travel time to these locations, ensuring that suggestions for ingredient procurement are not only based on proximity but also on the practicality of obtaining these ingredients at the user's convenience. For example, if a particular ingredient is available but only at a location that is closed at the time of cooking, the device may suggest a viable alternative that can be obtained more readily. Additionally, in scenarios where an ingredient is scarce in nearby locations, computing device 104 may be capable of suggesting substitutions based on similarity in flavor, texture, or culinary use, thus maintaining the integrity of the recipe while adapting to the constraints imposed by ingredient proximity and availability. This holistic approach to assessing ingredient accessibility not only enhances the user's cooking experience but also streamlines the meal preparation process by aligning it with real-world logistics and user-specific conditions.

With continued reference to FIG. 1, ingredient accessibility may include a consideration of ingredient price. Pricing can significantly influence the accessibility of ingredients, impacting the user's ability to purchase and use them in recipes. In an embodiment, computing device 104 may incorporate real-time pricing data into its evaluation process. This data could be sourced from various retail databases, online marketplaces, or direct feeds from grocery stores and supermarkets. By integrating price tracking functionalities, computing device 104 can analyze trends and fluctuations in ingredient costs over time. For example, if a user is planning a meal that includes avocados, computing device 104 might access historical and current pricing data to determine if avocados are financially reasonable at the time or if a more cost-effective substitute should be suggested. Additionally, computing device 104 may be configured to consider user-defined budget constraints. In this scenario, users can set a maximum budget for their recipes or grocery lists, and computing device 104 will prioritize ingredients and substitutes based on this financial parameter. If certain ingredients exceed the budget due to seasonal scarcity or increased demand, the device may offer alternatives that fit within the user's price range, thus ensuring the recipe remains affordable. In an additional embodiment, computing device 104 could leverage promotional and discount information from local retailers. By doing so, it can suggest purchasing strategies that might include buying in bulk during sales or choosing similar but cheaper ingredients when discounts are available. This approach not only enhances the accessibility of ingredients but also optimizes the cost-effectiveness of meal planning and grocery shopping, making it easier for users to manage their food expenses while still enjoying a diverse and satisfying diet.

With continued reference to FIG. 1, ingredient accessibility may be quantified by as an accessibility score. As used in the current disclosure, an “accessibility score” is quantification of the accessibility of each ingredient of the plurality of ingredients. This score may be designed to provide a quantification reflecting the overall case of obtaining specific ingredients, taking into account factors such as availability, price, proximity, and user preferences. In an embodiment, computing device 104 may calculate an accessibility score by aggregating data across multiple dimensions. In a non-limiting example, the score might be computed from a combination of availability, price, proximity, user preference. Availability may measure whether the ingredient is in stock at nearby locations. This factor may be weighted according to the criticality of the ingredient to the recipe. Price may be an evaluation of the affordability relative to the user's budget constraints. Ingredients priced within a user-defined range score higher. Proximity may be considered the distance to the nearest store carrying the ingredient. Closer locations yield higher scores, adjusted for travel feasibility and user mobility constraints. User Preference may incorporate user-specific preferences such as store type, brand loyalty, or organic versus non-organic products. Ingredients that the user already has in their position may be awarded extremely favorable accessibility scores especially in the categories of availability, price, and proximity.

With continued reference to FIG. 1, each factor of availability, price, proximity, user preference may be assigned a weight based on its importance to the user, and computing device 104 uses these weighted factors to generate a composite score. For example, if a user places a high value on organic ingredients, the availability of organic options would be weighted more heavily in the score calculation. Furthermore, computing device 104 could dynamically adjust these weights based on contextual factors, such as the urgency of the recipe (e.g., for immediate meal preparation versus future planning). The device could also update the score in real-time, reflecting changes in the market such as new stock arrivals, price changes, or updated user preferences. This accessibility Score may enable computing device 104 to provide tailored recommendations. For instance, if the score falls below a certain threshold, the device might suggest alternative recipes or substitute ingredients that have higher accessibility scores. This scoring system thus enhances the practicality and satisfaction of the user's cooking experience by ensuring that ingredient suggestions are realistic and attainable given their specific circumstances and constraints.

With continued reference to FIG. 1, a high accessibility score may indicate an ingredient that the user has or can easily obtain. Conversely, a low accessibility score could signal an ingredient that is virtually impossible for the user to obtain. Machine learning models can play a crucial role in generating accessibility scores by learning from patterns and correlations in large datasets. These models can automate the quantification of the accessibility of ingredients and continuously refine the scoring process based on new data and feedback. The accessibility scores may be normalized or standardized to ensure comparability across different datasets or variables. This means that the score is often scaled to fall within a specific range (i.e., 0 to 1 or −1 to 1). Normalization techniques can include min-max scaling, z-score normalization, or logarithmic transformation. In an embodiment, an accessibility score may be expressed as a numerical score, a linguistic value, or an alphabetical score. A non-limiting example, of a numerical score, may include a scale from 1-10, 1-100, 1-1000, and the like. In another non-limiting example, linguistic values may include, “Inaccessible,” “Moderately Accessible,” “Highly Accessible,” and the like. In some embodiments, linguistic values may correspond to a linguistic variable score range. For example, ingredient accessibility that receives a score between 40-60, on a scale from 1-100, may be considered the linguistic value “Moderately Accessible.”

With continued reference to FIG. 1, computing device 104 may generate an accessibility Score for each ingredient. Computing device 104 filter the list of ingredients as function of a comparison of the ingredients to a predefined threshold. This threshold may act as a benchmark to determine whether ingredients are sufficiently accessible to be included in a recipe. If the score for a particular ingredient falls below this threshold, indicating challenges in availability, cost, proximity, or alignment with user preferences, the ingredient may be deemed unsuitable for the current cooking context. Consequently, computing device 104 may filter out these lower-scoring ingredient identifiers from the recipe selection process. This filtering mechanism may ensure that only ingredients meeting or exceeding the accessibility threshold are presented to the user, streamlining the cooking process, and increasing the likelihood of a successful and satisfying culinary experience. Computing device 104 may be configured to generate an accessibility score for each ingredient within the lexicon of ingredients.

With continued reference to FIG. 1, computing device 104 is configured to compare first ingredient impact 132 to target profile 108. Comparing may include determining how well first ingredient impact 132 meets the needs and/or requirements of a user as specified within target profile 108. For instance, and without limitation, target profile 108 may specify that a user requires 60 grams of protein per day. In such an instance, computing device 104 may compare the 60 gram of protein per day requirement to first ingredient impact 132, which may specify that an ingredient such as one tablespoon of almond butter contains 4 grams of protein. Computing device 104 may accept and/or reject first ingredient identifier 124, based on the comparison. For instance and without limitation, a target profile 108 that specifies a user requires 10,000 milligrams of Vitamin C per day may be compared to first ingredient impact 132 which specifies that an ingredient such as an apple contains 80 milligrams of Vitamin C per serving, which may be rejected, in contrast to an ingredient such as guava which contains 600 milligrams of Vitamin C per serving. Comparing may include evaluating a temporal element of a nutrient anomaly, to determine a dose and/or quantity of an ingredient that may meet requirements contained within target profile 108. For instance and without limitation, a nutrient anomaly that contains a temporal element indicating that a user requires 15 grams of extra protein each day for the next three weeks while training for a marathon, may be utilized by computing device 104 to select and/or compare a first ingredient impact 132 that contains a higher dose of protein per serving as compared to a first ingredient impact 132 that contains a lower dose of protein per serving. In such an instance, computing device 104 may compare an ingredient such as tofu containing 20 grams of protein per serving to a target profile 108 requiring the user to consume a minimum of 15 grams of protein three times per day. Comparing may include determining by computing device 104 what serving size and/or portion of first ingredient identifier 124 may be recommended for a user based on a user's target profile 108. For instance and without limitation, comparing may include determining that a first ingredient identifier 124 may meet a user's nutritional targets contained within target profile 108 at a serving size of six ounces, however the first ingredient identifier 124 may not meet a user's target profile 108 at a larger serving size, and as such may even be determinantal to a user's health at a larger portion size. For instance and without limitation, a target profile 108 may specify that a user is to consume no more than 12 grams of mannitol per day, and as such, computing device 104 may suggest that a user is allowed no more than a serving size of 4 button mushrooms per day, because button mushrooms contain high quantities of mannitol. In such an instance, computing device 104 may recommend a user to avoid other ingredient identifiers that contain high levels of mannitol, including for example, celery, olives, onion, and pumpkin.

With continued reference to FIG. 1, computing device 104 is configured to generate a personal recipe 136. A “personal recipe,” as used in this disclosure, is a custom set of instructions for preparing a particular dish, which may include a list of ingredients and/or quantities of ingredients to be utilized to prepare the particular dish. A personal recipe 136 may be customized and/or optimized for each individual user. Personal recipe 136 may be generated as a function of first semantic unit and second semantic unit. This may be performed utilizing language processing as described below in more detail. Customization may include adjusting ingredients, ingredient quantities, cooking styles, and the like based on a user's target profile 108 and/or other individual and custom information relating to a user. In an embodiment, such information utilized to create and/or customize a personal recipe may be contained within ingredient database 128 and/or user database 112. For instance, and without limitation, a personal recipe 136 for a first user with a target profile 108 specifying that a user is allowed to consume no more than 1000 milligrams of sodium per day may be used to customize a personal recipe 136 for shrimp stir fry to adjust a quantity of a first ingredient identifier 124 containing soy sauce to be reduced to use no more than 1 tablespoon of low sodium soy sauce, as compared to a user profile 108 that does not contain a restriction on sodium, whereby the personal recipe 136 for shrimp stir fry may call for 2 tablespoons of regular soy sauce. Information pertaining to personal recipes 136 may be contained within recipe database 140. Recipe database may be implemented as any data structure suitable for use as user database 112 as described above in more detail. Generating a personal recipe 136 may include selecting a cooking style. A “cooking style,” as used in this disclosure, is a method of preparing a recipe. A cooking style may identify one or more styles such as grilling, steaming, baking, roasting, poaching, braising, boiling, searing, simmering, frying, barbequing, brining, basting, charbroiling, deglazing, marinating, pan frying, sauteing, stewing, and the like. In an embodiment, a personal recipe 136 may identify one or more cooking styles. For example, a personal recipe 136 for flank steak may include marinating the steak followed by grilling the steak. Computing device 104 may select a cooking style using target profile 108. Computing device 104 may utilize information contained within ingredient database 128.

With continued reference to FIG. 1, computing device 104 may select a cooking style as a function of a temporal component that considers the time required to gather ingredients and complete the cooking process. As used in this disclosure, a “temporal component” refers to the time it takes to gather and prepare the desired recipe. For instance, if a user has limited time, computing device 104 might select a cooking style like sautéing or pan-frying, which typically require shorter preparation and cooking times. Conversely, if the user plans for a weekend meal and has more time to dedicate, the device may suggest a cooking style such as marinating followed by grilling, which involves longer preparation times but potentially richer flavors. Computing device 104 may utilize data from the ingredient database 128 to estimate the time needed to procure all necessary ingredients. This estimation may evaluate factors such as the availability of ingredients in local stores, seasonal influences on ingredient availability, and any known delays in sourcing specific items. The cooking time for each style is also calculated based on typical durations associated with each cooking method, which are stored within the system. By integrating these temporal components, computing device 104 can provide a more tailored cooking experience that respects the user's schedule constraints. This thoughtful selection process not only ensures that the meal preparation is feasible within the given timeframe but also enhances the overall satisfaction with the cooking and dining experience, making it both enjoyable and practical.

With continued reference to FIG. 1, computing device 104 may generate personal recipe 136 by locating a second ingredient identifier 144. A second ingredient identifier 144 may include any ingredient suitable for use as first ingredient identifier 124 as described above in more detail. A second ingredient identifier 144 contains a second ingredient impact 148. A second ingredient impact 148 includes any ingredient impact suitable for use as first ingredient impact 132 as described above in more detail. In an embodiment, a second ingredient identifier 144 may be located using first ingredient identifier 124. In an embodiment, a first ingredient identifier 124 may be best suited to be matched and prepared in combination with a second ingredient identifier 144. For example, a first ingredient such as wild salmon may be best prepared in combination with a second ingredient such as lemon, while a first ingredient such as mahi mahi may be best prepared in combination with a second ingredient such as coconut. Information pertaining to palatability and ingredient combinations may be best stored within recipe database 140. In an embodiment, information pertaining to which ingredients may be substituted for one another due to allergies, intolerances, dislikes, and the like may be stored and contained within ingredient database 128. For example, information contained within ingredient database 128 may specify that a first ingredient such as fresh parsley may be substituted with basil, oregano, and/or chives such as when a user cannot consume parsley and/or if parsley may adversely affect a target profile 108. Second ingredient identifier 144 includes a second semantic unit. Second semantic unit may include any textual data suitable for use as first semantic unit as described above in more detail. Computing device 104 may locate a second ingredient identifier 144 by locating an ingredient that may improve a first ingredient impact 132. An ingredient may improve a first ingredient impact 132, such as when an ingredient may improve the nutrient profile of a first ingredient identifier 124 such as by impacting nutrient bioavailability, nutrient density, quantity and variety of nutrients, nutrient absorption and the like. Information pertaining to how an ingredient may improve a first ingredient impact 132 may be stored and contained within ingredient database 128. For instance, and without limitation, an ingredient such as olive oil may improve a first ingredient impact 132 of a tomato, whereby olive oil may boost absorption of lycopene contained within the tomato. In yet another non-limiting example, an ingredient such as avocado may improve a first ingredient impact 132 of leafy greens whereby monounsaturated fat found in an avocado may increase absorption and bioavailability of phytochemicals contained within leafy greens. Computing device 104 may determine that an ingredient may improve a first ingredient impact 132 such as by retrieving information that may be contained within ingredient database 128. In yet another non-limiting example, computing device 104 may determine how a particular ingredient such as a second ingredient identifier 144 may impact and/or improve a first ingredient impact 132 such as by using a machine-learning process, including any of the machine-learning processes as described herein. For instance and without limitation, computing device 104 may utilize a plurality of ingredients and first ingredient identifier 124 and first ingredient impact 132 as inputs to a machine-learning process and output which ingredients from the plurality of input ingredients are able to improve first ingredient impact 132. This may be performed utilizing any methodology as described herein.

With continued reference to FIG. 1, computing device 104 is configured to compare second ingredient impact 148 to first ingredient impact 132 and target profile 108. For instance and without limitation, computing device 104 may compare a second ingredient impact 148 for an ingredient such as kale to see how the second ingredient impact 148 may compare to and/or affect a first ingredient impact 132 for an ingredient such as chicken. In such an instance, computing device 104 may evaluate first ingredient impact 132 and second ingredient impact 148 in comparison to target profile 108, to determine how each ingredient impact will affect target profile 108. For instance and without limitation, a first ingredient such as a wild salmon may be compared to a second ingredient such as sweet potato and whereby both ingredients are compared to a target profile 108, to determine how each ingredient alone and/or in combination may affect each ingredient impact and/or impact target profile 108. Computing device 104 generates a personal recipe 136 as a function of first ingredient impact 132 and second ingredient impact 148.

With continued reference to FIG. 1, computing device 104 may generate personal recipe 136 using a machine learning process 152. A “machine learning process,” as used in this disclosure, is a process that automatically uses training data 156 to generate an algorithm that will be performed by computing device 104 to produce outputs such as personal recipe 136 given data provided as inputs such as target profile 108; this is in contrast to a non-machine learning software program where the commands to be executed are determined in advance by a user and written in a programming language.

Still referring to FIG. 1, “training data,” as used herein, is data containing correlations that a machine-learning process may use to model relationships between two or more categories of data elements. For instance, and without limitation, training data 156 may include a plurality of data entries, each entry representing a set of data elements that were recorded, received, and/or generated together; data elements may be correlated by shared existence in a given data entry, by proximity in a given data entry, or the like. Multiple data entries in training data 156 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 156 according to various correlations; correlations may indicate causative and/or predictive links between categories of data elements, which may be modeled as relationships such as mathematical relationships by machine-learning processes as described in further detail below. Training data 156 may be formatted and/or organized by categories of data elements, for instance by associating data elements with one or more descriptors corresponding to categories of data elements. As a non-limiting example, training data 156 may include data entered in standardized forms by persons or processes, such that entry of a given data element in a given field in a form may be mapped to one or more descriptors of categories. Elements in training data 156 may be linked to descriptors of categories by tags, tokens, or other data elements; for instance, and without limitation, training data 156 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 1, training data 156 may include one or more elements that are not categorized; that is, training data 156 may not be formatted or contain descriptors for some elements of data. Machine-learning algorithms and/or other processes may sort training data 156 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 156 to be made applicable for two or more distinct machine-learning algorithms as described in further detail below. Training data 156 used by machine learning process 152 may correlate any input data as described in this disclosure to any output data as described in this disclosure. As a non-limiting illustrative example an input such as a target profile 108 may be correlated to an output such as personal recipe 136. In yet another non-limiting example, an input such as a first ingredient impact 132 may be correlated to an output such as personal recipe 136.

With continued reference to FIG. 1, personal recipe 136 is generated as a function of first semantic unit and second semantic unit. In an embodiment, this may be performed utilizing a language processing module. Language processing module may be configured to extract, from the one or more documents, one or more words. One or more words may include, without limitation, strings of one or more characters, including without limitation any sequence or sequences of letters, numbers, punctuation, diacritic marks, engineering symbols, geometric dimensioning and tolerancing (GD&T) symbols, chemical symbols and formulas, spaces, whitespace, and other symbols, including any symbols usable as textual data as described above. Textual data may be parsed into tokens, which may include a simple word (sequence of letters separated by whitespace) or more generally a sequence of characters as described previously. The term “token,” as used herein, refers to any smaller, individual groupings of text from a larger source of text; tokens may be broken up by word, pair of words, sentence, or other delimitation. These tokens may in turn be parsed in various ways. Textual data may be parsed into words or sequences of words, which may be considered words as well. Textual data may be parsed into “n-grams”, where all sequences of n consecutive characters are considered. Any or all possible sequences of tokens or words may be stored as “chains”, for example for use as a Markov chain or Hidden Markov Model.

Still referring to FIG. 1, language processing module may compare extracted words to categories of physiological data recorded at diagnostic engine, one or more prognostic labels recorded at diagnostic engine, and/or one or more categories of prognostic labels recorded at diagnostic engine; such data for comparison may be entered on diagnostic engine as described above using expert data inputs or the like. In an embodiment, one or more categories may be enumerated, to find total count of mentions in such documents. Alternatively, or additionally, language processing module may operate to produce a language processing model. Language processing model may include a program automatically generated by computing device and/or language processing module to produce associations between one or more words extracted from at least a document and detect associations, including without limitation mathematical associations, between such words. Associations between language elements, where language elements include for purposes herein extracted words, relationships of such categories to other such term may include, without limitation, mathematical associations, including without limitation statistical correlations between any language element and any other language element and/or language elements. Statistical correlations and/or mathematical associations may include probabilistic formulas or relationships indicating, for instance, a likelihood that a given extracted word indicates a given category of semantic meaning. As a further example, statistical correlations and/or mathematical associations may include probabilistic formulas or relationships indicating a positive and/or negative association between at least an extracted word and/or a given semantic meaning; positive or negative indication may include an indication that a given document is or is not indicating a category semantic meaning. Whether a phrase, sentence, word, or other textual element in a document or corpus of documents constitutes a positive or negative indicator may be determined, in an embodiment, by mathematical associations between detected words, comparisons to phrases and/or words indicating positive and/or negative indicators that are stored in memory at computing device, or the like.

Still referring to 1, language processing module and/or diagnostic engine may generate the language processing model by any suitable method, including without limitation a natural language processing classification algorithm; language processing model may include a natural language process classification model that enumerates and/or derives statistical relationships between input term and output terms. Algorithm to generate language processing model may include a stochastic gradient descent algorithm, which may include a method that iteratively optimizes an objective function, such as an objective function representing a statistical estimation of relationships between terms, including relationships between input terms and output terms, in the form of a sum of relationships to be estimated. In an alternative or additional approach, sequential tokens may be modeled as chains, serving as the observations in a Hidden Markov Model (HMM). HMMs as used herein are statistical models with inference algorithms that that may be applied to the models. In such models, a hidden state to be estimated may include an association between an extracted word category of physiological data, a given relationship of such categories to prognostic labels, and/or a given category of prognostic labels. There may be a finite number of categories to which an extracted word may pertain; an HMM inference algorithm, such as the forward-back ward algorithm or the Viterbi algorithm, may be used to estimate the most likely discrete state given a word or sequence of words. Language processing module may combine two or more approaches. For instance, and without limitation, machine-learning program may use a combination of Naive-Bayes (NB), Stochastic Gradient Descent (SGD), and parameter grid-searching classification techniques; the result may include a classification algorithm that returns ranked associations.

Continuing to refer to FIG. 1, generating language processing model may include generating a vector space, which may be a collection of vectors, defined as a set of mathematical objects that can be added together under an operation of addition following properties of associativity, commutativity, existence of an identity element, and existence of an inverse element for each vector, and can be multiplied by scalar values under an operation of scalar multiplication compatible with field multiplication, and that has an identity element is distributive with respect to vector addition, and is distributive with respect to field addition. Each vector in an n-dimensional vector space may be represented by an n-tuple of numerical values. Each unique extracted word and/or language element as described above may be represented by a vector of the vector space. In an embodiment, each unique extracted and/or other language element may be represented by a dimension of vector space; as a non-limiting example, each element of a vector may include a number representing an enumeration of co-occurrences of the word and/or language element represented by the vector with another word and/or language element. Vectors may be normalized, scaled according to relative frequencies of appearance and/or file sizes. In an embodiment associating language elements to one another as described above may include computing a degree of vector similarity between a vector representing each language element and a vector representing another language element; vector similarity may be measured according to any norm for proximity and/or similarity of two vectors, including without limitation cosine similarity, which measures the similarity of two vectors by evaluating the cosine of the angle between the vectors, which can be computed using a dot product of the two vectors divided by the lengths of the two vectors. Degree of similarity may include any other geometric measure of distance between vectors.

Still referring to FIG. 1, language processing module may use a corpus of documents to generate associations between language elements in a language processing module, and diagnostic engine may then use such associations to analyze words extracted from one or more documents and determine that the one or more documents indicate significance of a category. In an embodiment, diagnostic engine may perform this analysis using a selected set of significant documents, such as documents identified by one or more experts as representing good information; experts may identify or enter such documents via graphical user interface, or may communicate identities of significant documents according to any other suitable method of electronic communication, or by providing such identity to other persons who may enter such identifications into diagnostic engine. Documents may be entered into a computing device by being uploaded by an expert or other persons using, without limitation, file transfer protocol (FTP) or other suitable methods for transmission and/or upload of documents; alternatively or additionally, where a document is identified by a citation, a uniform resource identifier (URI), uniform resource locator (URL) or other datum permitting unambiguous identification of the document, diagnostic engine may automatically obtain the document using such an identifier, for instance by submitting a request to a database or compendium of documents such as JSTOR as provided by Ithaka Harbors, Inc. of New York.

With continued reference to FIG. 1, computing device 104 may be configured to classify the cooking style and the personal recipe 132 into distinct categories to enhance the customization and relevance of recipe suggestions. This classification process involves two primary types of categories: meal categories and cuisine categories. As used in the current disclosure, “meal categories” refer to classifications based on the type of meal for which the personal recipe is intended. These categories help tailor the recipe to fit specific times of the day or dietary patterns, enhancing the user's meal planning. Examples of meal categories include breakfast, lunch, dinner, snacks, and late-night meals. Each category can be further subdivided based on other factors such as the meal's nutritional content, its purpose (e.g., weight loss, high energy, low carb), and typical consumption context (e.g., quick breakfasts, family dinners). This granular classification allows computing device 104 to suggest recipes that align closely with the user's daily eating schedule and nutritional goals.

With continued reference to FIG. 1, classification process may involve classifying the cooking style and the personal recipe 132 into cuisine categories. As used in the current disclosure, “cuisine categories” are used to classify recipes based on cultural or geographic culinary traditions. This classification allows the device to consider the cultural origins of a recipe, which can influence the ingredients used, cooking techniques, and the overall flavor profile of the dish. Cuisine categories might include, but are not limited to, Indian, Chinese, Mexican, Cajun, Southern, Greek, and African cuisines. Each category represents a distinct culinary tradition and includes a variety of sub-categories that might focus on regional differences within the broader category (e.g., Northern versus Southern Italian cuisine).

With continued reference to FIG. 1, computing device 104 may employ machine learning (ML) models and classifiers to categorize both the cooking style and the personal recipe 132 into specific meal and cuisine categories. Computing device 104 may process the ingredients, cooking methods, and the intended meal time provided by the user to facilitate this classification. By employing ML techniques, computing device 104 can identify patterns and draw parallels with existing categorized recipes, enabling it to accurately assign a new recipe into the correct meal and cuisine categories. This may be done to enhance the device's ability to offer recipe suggestions that are not only timely—appropriate for the specific part of the day—but also deeply resonant with the user's cultural preferences or specific dietary restrictions tied to particular cuisines. Additionally, this intelligent classification system may be used to deliver a customized cooking experience by suggesting adjustments to recipes based on the user's individual preferences within these categories. For instance, if a user has a preference for low-calorie meals, the device can modify a traditional recipe from any selected cuisine category to meet this dietary requirement, all while preserving the authentic flavors and techniques characteristic of the original cuisine.

With continued reference to FIG. 1, computing device 104 is configured to generate a recipe depiction as a function of the cooking style and the personal recipe 132 preferences of the user. As used in the current disclosure, “recipe depiction” refers to a comprehensive visual and textual representation of a recipe, tailored to individual preferences and dietary needs. This depiction includes visual elements such as images or videos of the prepared dish, demonstrating both the final product and the steps involved in creating it according to the personal recipe 136 in the specified cooking style. In an embodiment, the recipe depiction may include an instructional video. As used in the current disclosure, an “instructional video” is a video that provides instructions about how to prepare the personal recipe in the selected cooking style. Computing device 104 may generate the recipe depiction or instructional video by analyzing the user's input concerning their preferred cooking style—be it fried, baked, sauté, vegan, low-calorie, or any other specified style—and the personal modifications they wish to incorporate into the recipe. Based on this data, the computing device 104 may select the appropriate ingredients from a database, adjusting traditional recipes to fit the user's nutritional and dietary needs and preferences. The visual components of the recipe depiction may be generated using either stored images and videos from a comprehensive culinary database or newly created content that specifically matches the cooking instructions of the adapted recipe. For example, if the user's modified recipe requires a technique not commonly used, such as sous-vide cooking, computing device 104 might include a video tutorial on how to set up and use a sous-vide machine. Furthermore, the textual component of the recipe depiction includes a detailed list of ingredients, customized cooking instructions, and any necessary preparatory steps formatted in a user-friendly manner. These instructions may be dynamically adjusted to reflect the specific ingredients and techniques required by the cooking style and personal adjustments. Additionally, the recipe depiction might also provide tips for garnishing, serving suggestions, and alternatives for potential ingredient substitutions if the original ingredients are not accessible.

With continued reference to FIG. 1, computing device 104 may generate the recipe depiction, including images and video content, to assist users in preparing meals according to their specified cooking style and personal recipe preferences. Images and videos that are included in the recipe depiction, computing device 104 might utilize a database of pre-existing culinary media. This database may be extensive, containing high-quality images and videos of a wide variety of dishes, cooking techniques, and ingredient preparations. When a user selects a recipe or inputs their personal modifications, computing device 104 may search this database to find the best visual matches that illustrate the necessary steps and the final dish. These visuals may be selected based on their relevance to the specific ingredients and cooking methods detailed in the user's recipe. If the database lacks specific content, or if the user's modifications are unique, computing device 104 can engage in generating new visual content. Computing device 104 may employ a culinary machine learning models to generate synthetic images and videos that closely mimic real-life cooking scenarios. These AI-generated visuals may be created by analyzing thousands of culinary images and videos to produce new content that accurately represents the cooking instructions and final dishes. In an embodiment, computing device 104 may integrate interactive elements into the video content, such as clickable overlays that provide additional information about specific ingredients or cooking techniques shown in the video. For instance, a user watching a video on how to knead dough might be able to click on an overlay to get tips on achieving the perfect consistency or alternatives for if the dough is too sticky. The device may also incorporate user feedback to improve the relevance and quality of the images and videos. Users can rate the helpfulness of the visuals, and computing device 104 uses this data to refine its search algorithms and content generation processes, ensuring that future recipe depictions are more closely aligned with user needs and preferences.

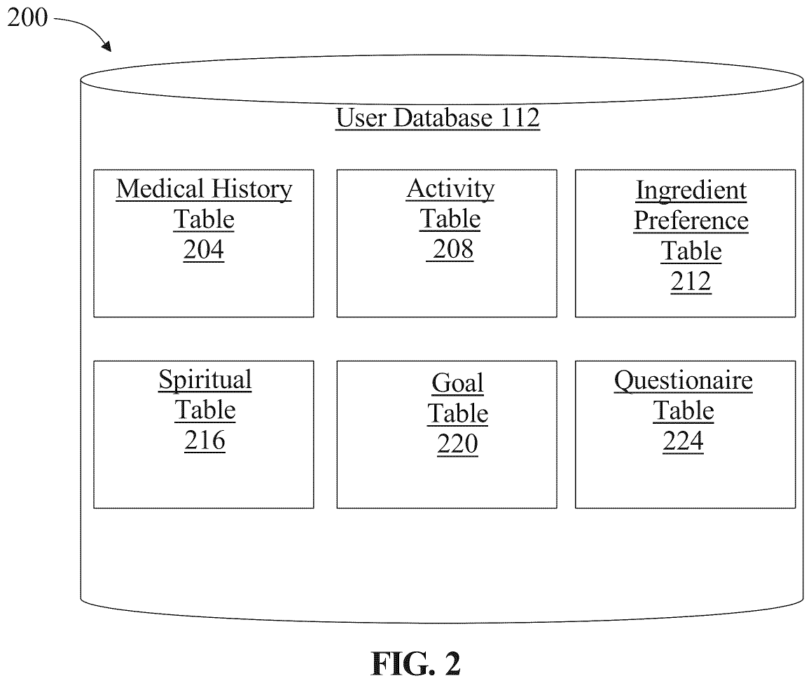

With continued reference to FIG. 1, processor 104 may generate recipe depiction using a depiction machine-learning model. As used in the current disclosure, a “depiction machine-learning model” is a machine-learning model that is configured to generate recipe depiction. Depiction machine-learning model may be consistent with the machine-learning model described below in FIG. 2. Inputs to the depiction machine-learning model may include personal recipe 136, cooking style, target profile, nutrient anomaly, examples of recipe depiction, and the like. Outputs to the depiction machine-learning model may include recipe depiction tailored to the personal recipe 136 and cooking style. Depiction training data may include a plurality of data entries containing a plurality of inputs that are correlated to a plurality of outputs for training a processor by a machine-learning process. In an embodiment, depiction training data may include a plurality of personal recipe 136 and cooking style correlated to examples of recipe depiction. Depiction training data may be received from database 300. Depiction training data may contain information about personal recipe 136, cooking style, target profile, nutrient anomaly, examples of recipe depiction, and the like. In an embodiment, depiction training data may be iteratively updated as a function of the input and output results of past depiction machine-learning model or any other machine-learning model mentioned throughout this disclosure. The machine-learning model may be performed using, without limitation, linear machine-learning models such as without limitation logistic regression and/or naive Bayes machine-learning models, nearest neighbor machine-learning models such as k-nearest neighbors machine-learning models, support vector machines, least squares support vector machines, fisher's linear discriminant, quadratic machine-learning models, decision trees, boosted trees, random forest machine-learning model, and the like.

With continued reference to FIG. 1, computing device 104 can utilize a depiction machine-learning model to generate recipe depictions. This machine learning model may be trained to analyze, interpret, and generate culinary content, such as images and videos, which align with user-defined recipes and cooking styles. The training and generation process of this model may involve multiple stages designed to optimize the model's effectiveness in producing accurate and useful culinary depictions. Generating depiction training data may include gathering a large and diverse dataset that includes a wide range of culinary images, videos, recipes, and their corresponding cooking instructions. This data may be sourced from various culinary websites, cookbooks, and existing databases. It may also include user-generated content, which may provide numerous culinary practices and preferences. The diversity in the dataset may ensure that the model can learn and later replicate a wide array of cooking styles and techniques. Once collected, the depiction training data may be prepared and preprocessed. This may include labeling the images and videos with metadata that describe the content, such as the type of dish, the ingredients used, and the cooking techniques depicted. Videos may be segmented into shorter clips that focus on specific steps or techniques, and images may be categorized by cooking stages and final presentations. Textual data, such as recipes and instructions, may be processed using natural language processing techniques to extract relevant features like ingredient lists and cooking temperatures. Once the training data is prepared, the machine learning model may be trained using the gathered training data. This may involve using convolutional neural networks (CNNs) for image and video processing, combined with recurrent neural networks (RNNs) or transformers for analyzing and generating textual instructions. The model may be trained to recognize patterns and relationships between different types of data, such as the appearance of a dish based on its ingredients and cooking methods, or the sequence of steps required to achieve a certain culinary result. After training, the model may be tested using a separate set of data not included in the training phase. This testing helps identify any issues with the model's performance, such as inaccuracies in recognizing specific ingredients or difficulties in replicating certain cooking styles. Feedback from these tests is used to refine and retrain the model, improving its accuracy and reliability.

With continued reference to FIG. 1, machine learning plays a crucial role in enhancing the function of software for generating recipe depictions. This may include identifying patterns within the cooking style and the personal recipe 132 that lead to changes in the capabilities of the depiction machine-learning model. By analyzing vast amounts of images and videos related to cooking styles and the personal recipes 132, machine learning algorithms can identify patterns, correlations, and dependencies that contribute to the generation of depiction machine-learning model. These algorithms can extract valuable insights from various sources, including text, document, images, videos, and the like. By applying machine learning techniques, the software can generate recipe depictions extremely accurately and quickly. Machine learning models may enable the software to learn from past collaborative experiences of the entities and iteratively improve its training data over time.

With continued reference to FIG. 1, computing device 104 may be configured to update the depiction training data of the depiction machine-learning model using user inputs. A depiction machine-learning model may use user input to update its training data, thereby improving its performance, speed, and accuracy. In embodiments, the depiction machine-learning model may be iteratively updated using input and output results of past iterations of depiction machine-learning models. The depiction machine-learning model may then be iteratively retrained using the updated depiction training data. For instance, and without limitation, depiction machine-learning model may be trained using a first training data from, for example, and without limitation, training data from a user input or database. The depiction machine-learning model may then be updated by using previous inputs and outputs from the depiction machine-learning model as second set of training data to then retrain a newer iteration of depiction machine-learning model. This process of updating the depiction machine-learning model and its associated training data may be continuously done to create subsequent depiction machine-learning models that possess improved speed and accuracy relative to their predecessors. When users interact with the software, their actions, preferences, and feedback provide valuable information that can be used to refine and enhance the model. This user input is collected and incorporated into the training data, allowing the machine learning model to learn from real-world interactions and adapt its predictions accordingly. By continually incorporating user input, the model becomes more responsive to user needs and preferences, capturing evolving trends and patterns. This iterative process of updating the training data with user input enables the machine learning model to deliver more personalized and relevant results, ultimately enhancing the overall user experience. The discussion within this paragraph may apply to both the depiction machine-learning model and any other machine-learning model/classifier discussed herein.

Incorporating the user feedback may include updating the training data by removing or adding correlations of user data to a path or resources as indicated by the feedback. Any machine-learning model as described herein may have the training data updated based on such feedback or data gathered using any method described herein. For example, when correlations in training data are based on outdated information, a web crawler may update such correlations based on more recent resources and information.

With continued reference to FIG. 1, computing device 104 may use user feedback to train the machine-learning models and/or classifiers described above. For example, machine-learning models and/or classifiers may be trained using past inputs and outputs of the machine-learning model. In some embodiments, if user feedback indicates that an output of machine-learning models and/or classifiers was “unfavorable,” then that output and the corresponding input may be removed from training data used to train machine-learning models and/or classifiers, and/or may be replaced with a value entered by, e.g., another value that represents an ideal output given the input the machine learning model originally received, permitting use in retraining, and adding to training data; in either case, machine-learning models may be retrained with modified training data as described in further detail below. In some embodiments, training data of classifier may include user feedback.

With continued reference to FIG. 1, in some embodiments, an accuracy score may be calculated for the machine-learning model and/or classifier using user feedback. For the purposes of this disclosure, “accuracy score,” is a numerical value concerning the accuracy of a machine-learning model. For example, the accuracy/quality of the output depiction machine-learning model may be averaged to determine an accuracy score. In some embodiments, an accuracy score may be determined for the quality/accuracy of the recipe depiction. Accuracy score or another score as described above may indicate a degree of retraining needed for a machine-learning model and/or classifier. Computing device 104 may perform a larger number of retraining cycles for a higher number (or lower number, depending on a numerical interpretation used), and/or may collect more training data for such retraining. The discussion within this paragraph and the paragraphs preceding this paragraph may apply to both the depiction machine-learning model and/or any other machine-learning model/classifier mentioned herein.

Referring now to FIG. 2, an exemplary embodiment 200 of user database 112 is illustrated. User database 112 may be implemented as any data structure suitable for use as described above in more detail in reference to FIG. 1. One or more tables contained within user database 112 may include medical history table 204; medical history table 204 may include any information pertaining to a user's medical history. For instance, and without limitation, medical history table 204 may include an entry describing a chronic medical condition that the user may suffer from, such as rheumatoid arthritis. One or more tables contained within user database 112 may include activity table 208; activity table 208 may include any information relating to physical activity that the user may engage in. For instance, and without limitation, activity table 208 may describe a user's exercise routine, whereby the user may engage in strength training two days per week and cardiovascular exercise three days per week. One or more tables contained within user database 112 may include ingredient preference table 212; ingredient preference table 212 may include any information describing foods that a user likes, dislikes, avoids, abstains from consuming and the like. For instance, and without limitation, ingredient preference table 212 may specify that a user does not consume pork containing products for religious reasons. One or more tables contained within user database 112 may include spiritual table 216; spiritual table 216 may include any information describing a user's spiritual practice. For instance, and without limitation, spiritual table 216 may specify that a user engages in a meditation practice three days per week for fifteen minutes. One or more tables contained within user database 112 may include goal table 220; goal table 220 may include information describing any personal goals that a user seeks to achieve. For instance, and without limitation, goal table 220 contain an entry describing a user's wellness related goal to lose fifteen pounds over the next three months. One or more tables contained within user database 112 may include questionnaire table 224; questionnaire table 224 may include information about a user, obtained from a questionnaire. For instance and without limitation, questionnaire table 224 may include answers to questions that specify the eating patterns of a user, including the time of day a user consumes meals, how many meals per day the user typically cats, and how many snacks per day the user cats.

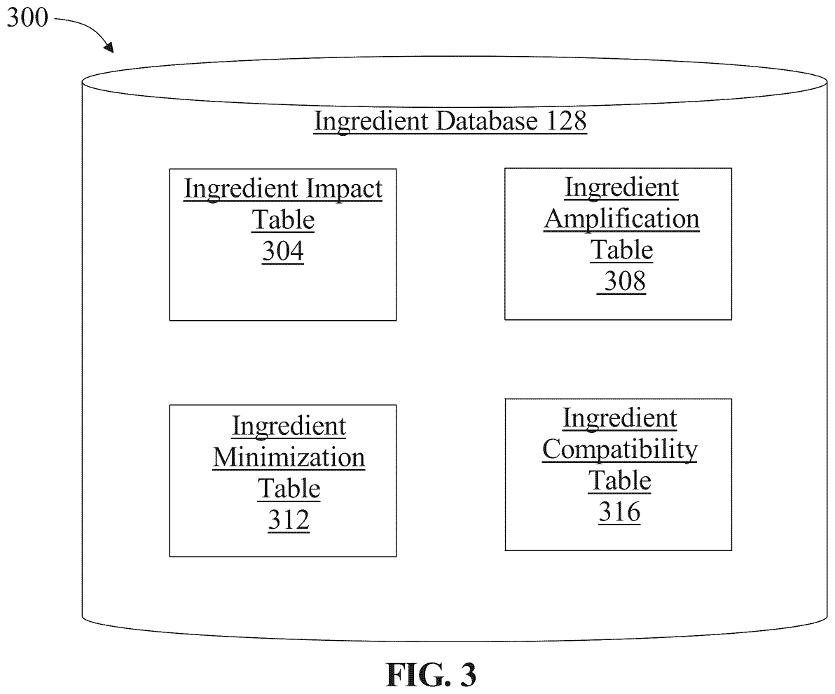

Referring now to FIG. 3, an exemplary embodiment 300 of ingredient database 128 is illustrated. Ingredient database 128 may be implemented as any data structure suitable for use as user database 112 as described above in more detail in reference to FIG. 1. One or more tables contained within ingredient database 128 may include ingredient impact table 304; ingredient impact table 304 may contain information pertaining to ingredient impacts for various ingredients. One or more tables contained within ingredient database 128 may include ingredient amplification table 308; ingredient amplification table 308 may contain information describing ingredients that may amplify ingredient impacts of other ingredients. One or more tables contained within ingredient database 128 may include ingredient minimization table 312; ingredient minimization table 312 may contain information describing ingredients that may minimize ingredient impacts of other ingredients. One or more tables contained within ingredient database 128 may include ingredient compatibility table 316; ingredient compatibility table 316 may include information describing the compatibility of various ingredients.

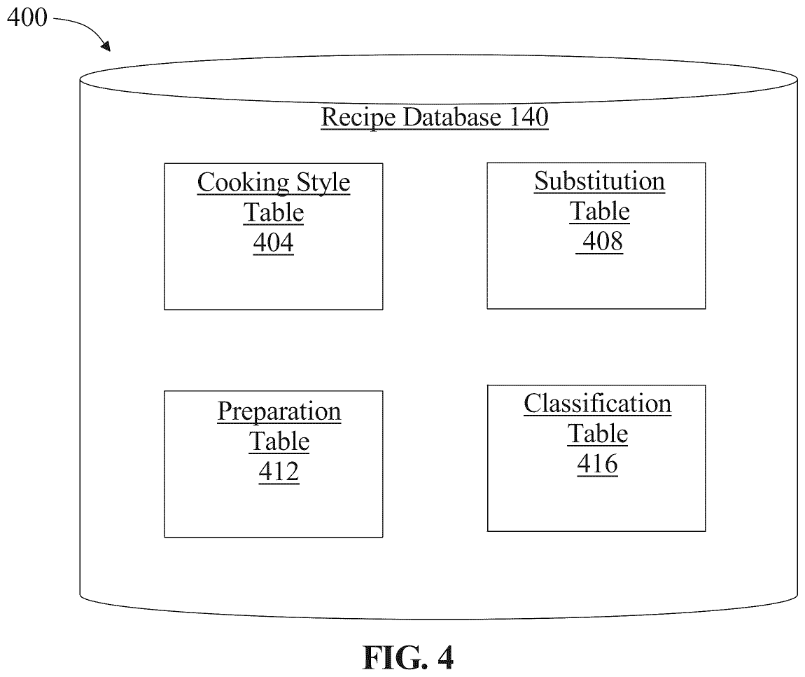

Referring now to FIG. 4, an exemplary embodiment 400 of recipe database 140 is illustrated. Recipe database 140 may be implemented as any data structure suitable for use as user database 112 as described above in more detail in reference to FIG. 1. One or more tables contained within recipe database 140 may include cooking style table 404; cooking style table 404 may contain information describing various cooking styles and/or cooking techniques that may be applied to various recipes. One or more tables contained within recipe database 140 may include substitution table 408; substitution table 408 may include information describing which ingredients can be substituted for one another in various recipes, due to allergies, dislikes, nutritional content, and the like. One or more tables contained within recipe database may include preparation table 412; preparation table 412 may include information describing various preparation techniques for recipes. One or more tables contained within recipe database 140 may include classification table 416; classification table 416 may contain information describing diets and/or styles of eating that a recipe may be suitable for.

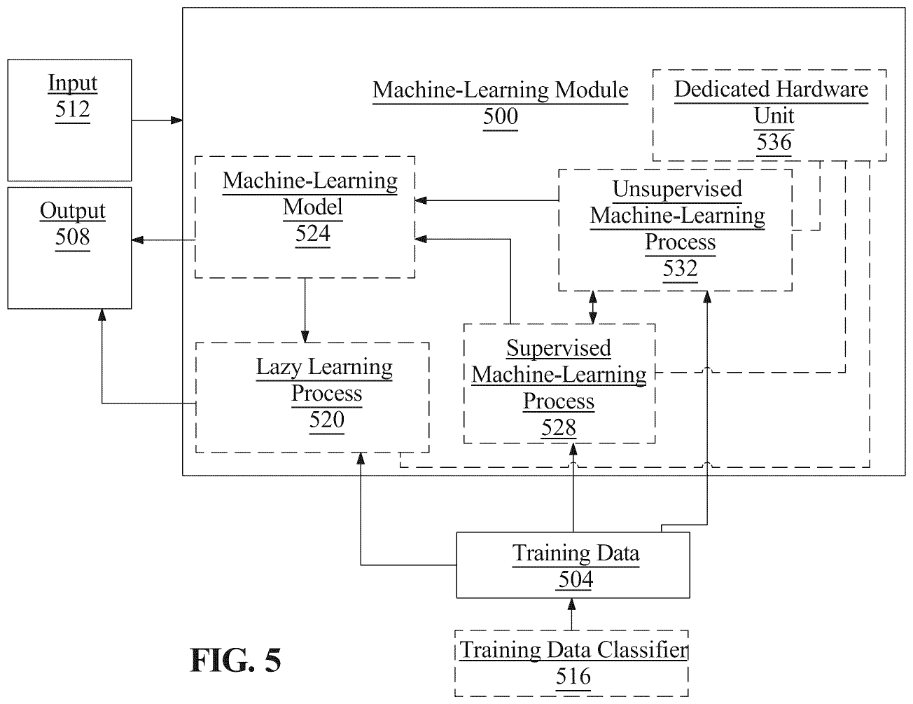

Referring now to FIG. 5, an exemplary embodiment of a machine-learning module 500 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 504 to generate an algorithm instantiated in hardware or software logic, data structures, and/or functions that will be performed by a computing device/module to produce outputs 508 given data provided as inputs 512; this is in contrast to a non-machine learning software program where the commands to be executed are determined in advance by a user and written in a programming language.

Still referring to FIG. 5, “training data,” as used herein, is data containing correlations that a machine-learning process may use to model relationships between two or more categories of data elements. For instance, and without limitation, training data 504 may include a plurality of data entries, also known as “training examples,” each entry representing a set of data elements that were recorded, received, and/or generated together; data elements may be correlated by shared existence in a given data entry, by proximity in a given data entry, or the like. Multiple data entries in training data 504 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 504 according to various correlations; correlations may indicate causative and/or predictive links between categories of data elements, which may be modeled as relationships such as mathematical relationships by machine-learning processes as described in further detail below. Training data 504 may be formatted and/or organized by categories of data elements, for instance by associating data elements with one or more descriptors corresponding to categories of data elements. As a non-limiting example, training data 504 may include data entered in standardized forms by persons or processes, such that entry of a given data element in a given field in a form may be mapped to one or more descriptors of categories. Elements in training data 504 may be linked to descriptors of categories by tags, tokens, or other data elements; for instance, and without limitation, training data 504 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 5, training data 504 may include one or more elements that are not categorized; that is, training data 504 may not be formatted or contain descriptors for some elements of data. Machine-learning algorithms and/or other processes may sort training data 504 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 504 to be made applicable for two or more distinct machine-learning algorithms as described in further detail below. Training data 504 used by machine-learning module 500 may correlate any input data as described in this disclosure to any output data as described in this disclosure. As a non-limiting illustrative example, examples of cooking styles and personal recipes 132 as inputs correlated to examples of recipe depictions as outputs.

Further referring to FIG. 5, training data may be filtered, sorted, and/or selected using one or more supervised and/or unsupervised machine-learning processes and/or models as described in further detail below; such models may include without limitation a training data classifier 516. Training data classifier 516 may include a “classifier,” which as used in this disclosure is a machine-learning model as defined below, such as a data structure representing and/or using a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. A distance metric may include any norm, such as, without limitation, a Pythagorean norm. Machine-learning module 500 may generate a classifier using a classification algorithm, defined as a processes whereby a computing device and/or any module and/or component operating thereon derives a classifier from training data 504. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 516 may classify elements of training data according to various ingredients and/or cooking styles.

Still referring to FIG. 5, Computing device may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)÷P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device may then calculate a likelihood table by calculating probabilities of different data entries and classification labels. Computing device may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 5, Computing device may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 5, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute l as derived using a Pythagorean norm:

$$l = \sqrt{{Σ}_{i = 0}^{n} {a}_{i}^{2}} ,$$

where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

With further reference to FIG. 5, training examples for use as training data may be selected from a population of potential examples according to cohorts relevant to an analytical problem to be solved, a classification task, or the like. Alternatively or additionally, training data may be selected to span a set of likely circumstances or inputs for a machine-learning model and/or process to encounter when deployed. For instance, and without limitation, for each category of input data to a machine-learning process or model that may exist in a range of values in a population of phenomena such as images, user data, process data, physical data, or the like, a computing device, processor, and/or machine-learning model may select training examples representing each possible value on such a range and/or a representative sample of values on such a range. Selection of a representative sample may include selection of training examples in proportions matching a statistically determined and/or predicted distribution of such values according to relative frequency, such that, for instance, values encountered more frequently in a population of data so analyzed are represented by more training examples than values that are encountered less frequently. Alternatively or additionally, a set of training examples may be compared to a collection of representative values in a database and/or presented to a user, so that a process can detect, automatically or via user input, one or more values that are not included in the set of training examples. Computing device, processor, and/or module may automatically generate a missing training example; this may be done by receiving and/or retrieving a missing input and/or output value and correlating the missing input and/or output value with a corresponding output and/or input value collocated in a data record with the retrieved value, provided by a user and/or other device, or the like.

Continuing to refer to FIG. 5, computer, processor, and/or module may be configured to preprocess training data. “Preprocessing” training data, as used in this disclosure, is transforming training data from raw form to a format that can be used for training a machine learning model. Preprocessing may include sanitizing, feature selection, feature scaling, data augmentation and the like.

Still referring to FIG. 5, computer, processor, and/or module may be configured to sanitize training data. “Sanitizing” training data, as used in this disclosure, is a process whereby training examples are removed that interfere with convergence of a machine-learning model and/or process to a useful result. For instance, and without limitation, a training example may include an input and/or output value that is an outlier from typically encountered values, such that a machine-learning algorithm using the training example will be adapted to an unlikely amount as an input and/or output; a value that is more than a threshold number of standard deviations away from an average, mean, or expected value, for instance, may be eliminated. Alternatively or additionally, one or more training examples may be identified as having poor quality data, where “poor quality” is defined as having a signal to noise ratio below a threshold value. Sanitizing may include steps such as removing duplicative or otherwise redundant data, interpolating missing data, correcting data errors, standardizing data, identifying outliers, and the like. In a nonlimiting example, sanitization may include utilizing algorithms for identifying duplicate entries or spell-check algorithms.

As a non-limiting example, and with further reference to FIG. 5, images used to train an image classifier or other machine-learning model and/or process that takes images as inputs or generates images as outputs may be rejected if image quality is below a threshold value. For instance, and without limitation, computing device, processor, and/or module may perform blur detection, and eliminate one or more Blur detection may be performed, as a non-limiting example, by taking Fourier transform, or an approximation such as a Fast Fourier Transform (FFT) of the image and analyzing a distribution of low and high frequencies in the resulting frequency-domain depiction of the image; numbers of high-frequency values below a threshold level may indicate blurriness. As a further non-limiting example, detection of blurriness may be performed by convolving an image, a channel of an image, or the like with a Laplacian kernel; this may generate a numerical score reflecting a number of rapid changes in intensity shown in the image, such that a high score indicates clarity, and a low score indicates blurriness. Blurriness detection may be performed using a gradient-based operator, which measures operators based on the gradient or first derivative of an image, based on the hypothesis that rapid changes indicate sharp edges in the image, and thus are indicative of a lower degree of blurriness. Blur detection may be performed using Wavelet-based operator, which takes advantage of the capability of coefficients of the discrete wavelet transform to describe the frequency and spatial content of images. Blur detection may be performed using statistics-based operators take advantage of several image statistics as texture descriptors in order to compute a focus level. Blur detection may be performed by using discrete cosine transform (DCT) coefficients in order to compute a focus level of an image from its frequency content.

Continuing to refer to FIG. 5, computing device, processor, and/or module may be configured to precondition one or more training examples. For instance, and without limitation, where a machine learning model and/or process has one or more inputs and/or outputs requiring, transmitting, or receiving a certain number of bits, samples, or other units of data, one or more training examples' elements to be used as or compared to inputs and/or outputs may be modified to have such a number of units of data. For instance, a computing device, processor, and/or module may convert a smaller number of units, such as in a low pixel count image, into a desired number of units, for instance by upsampling and interpolating. As a non-limiting example, a low pixel count image may have 100 pixels, however a desired number of pixels may be 128. Processor may interpolate the low pixel count image to convert the 100 pixels into 128 pixels. It should also be noted that one of ordinary skill in the art, upon reading this disclosure, would know the various methods to interpolate a smaller number of data units such as samples, pixels, bits, or the like to a desired number of such units. In some instances, a set of interpolation rules may be trained by sets of highly detailed inputs and/or outputs and corresponding inputs and/or outputs downsampled to smaller numbers of units, and a neural network or other machine learning model that is trained to predict interpolated pixel values using the training data. As a non-limiting example, a sample input and/or output, such as a sample picture, with sample-expanded data units (e.g., pixels added between the original pixels) may be input to a neural network or machine-learning model and output a pseudo replica sample-picture with dummy values assigned to pixels between the original pixels based on a set of interpolation rules. As a non-limiting example, in the context of an image classifier, a machine-learning model may have a set of interpolation rules trained by sets of highly detailed images and images that have been downsampled to smaller numbers of pixels, and a neural network or other machine learning model that is trained using those examples to predict interpolated pixel values in a facial picture context. As a result, an input with sample-expanded data units (the ones added between the original data units, with dummy values) may be run through a trained neural network and/or model, which may fill in values to replace the dummy values. Alternatively or additionally, processor, computing device, and/or module may utilize sample expander methods, a low-pass filter, or both. As used in this disclosure, a “low-pass filter” is a filter that passes signals with a frequency lower than a selected cutoff frequency and attenuates signals with frequencies higher than the cutoff frequency. The exact frequency response of the filter depends on the filter design. Computing device, processor, and/or module may use averaging, such as luma or chroma averaging in images, to fill in data units in between original data units.

In some embodiments, and with continued reference to FIG. 5, computing device, processor, and/or module may down-sample elements of a training example to a desired lower number of data elements. As a non-limiting example, a high pixel count image may have 256 pixels, however a desired number of pixels may be 128. Processor may down-sample the high pixel count image to convert the 256 pixels into 128 pixels. In some embodiments, processor may be configured to perform downsampling on data. Downsampling, also known as decimation, may include removing every Nth entry in a sequence of samples, all but every Nth entry, or the like, which is a process known as “compression,” and may be performed, for instance by an N-sample compressor implemented using hardware or software. Anti-aliasing and/or anti-imaging filters, and/or low-pass filters, may be used to clean up side-effects of compression.

Further referring to FIG. 5, feature selection includes narrowing and/or filtering training data to exclude features and/or elements, or training data including such elements, that are not relevant to a purpose for which a trained machine-learning model and/or algorithm is being trained, and/or collection of features and/or elements, or training data including such elements, on the basis of relevance or utility for an intended task or purpose for a trained machine-learning model and/or algorithm is being trained. Feature selection may be implemented, without limitation, using any process described in this disclosure, including without limitation using training data classifiers, exclusion of outliers, or the like.

With continued reference to FIG. 5, feature scaling may include, without limitation, normalization of data entries, which may be accomplished by dividing numerical fields by norms thereof, for instance as performed for vector normalization. Feature scaling may include absolute maximum scaling, wherein each quantitative datum is divided by the maximum absolute value of all quantitative data of a set or subset of quantitative data. Feature scaling may include min-max scaling, in which each value X has a minimum value Xₘᵢₙ in a set or subset of values subtracted therefrom, with the result divided by the range of the values, give maximum value in the set or subset

$${X}_{max} : {X}_{n e w} = \frac{X - {X}_{min}}{{X}_{max} - {X}_{min}} .$$

Feature scaling may include mean normalization, which involves use of a mean value of a set and/or subset of values, Xₘₑₐₙ with maximum and minimum values:

$${X}_{n e w} = \frac{X - {X}_{m e a n}}{{X}_{max} - {X}_{min}} .$$

Feature scaling may include standardization, where a difference between X and Xₘₑₐₙ is divided by a standard deviation o of a set or subset of values:

$${X}_{n e w} = \frac{X - {X}_{m e a n}}{σ} .$$

Scaling may be performed using a median value of a set or subset X_{median} and/or interquartile range (IQR), which represents the difference between the 25^{th} percentile value and the 50^{th} percentile value (or closest values thereto by a rounding protocol), such as:

$${X}_{n e w} = \frac{X - {X}_{m e d i a n}}{IQR} .$$

Persons skilled in the art, upon reviewing the entirety of this disclosure, will be aware of various alternative or additional approaches that may be used for feature scaling.

Further referring to FIG. 5, computing device, processor, and/or module may be configured to perform one or more processes of data augmentation. “Data augmentation” as used in this disclosure is addition of data to a training set using elements and/or entries already in the dataset. Data augmentation may be accomplished, without limitation, using interpolation, generation of modified copies of existing entries and/or examples, and/or one or more generative AI processes, for instance using deep neural networks and/or generative adversarial networks; generative processes may be referred to alternatively in this context as “data synthesis” and as creating “synthetic data.” Augmentation may include performing one or more transformations on data, such as geometric, color space, affine, brightness, cropping, and/or contrast transformations of images.

Still referring to FIG. 5, machine-learning module 500 may be configured to perform a lazy-learning process 520 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 504. Heuristic may include selecting some number of highest-ranking associations and/or training data 504 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 5, machine-learning processes as described in this disclosure may be used to generate machine-learning models 524. A “machine-learning model,” as used in this disclosure, is a data structure representing and/or instantiating a mathematical and/or algorithmic representation of a relationship between inputs and outputs, as generated using any machine-learning process including without limitation any process as described above, and stored in memory; an input is submitted to a machine-learning model 524 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 524 may be generated by creating an artificial neural network, such as a convolutional neural network comprising an input layer of nodes, one or more intermediate layers, and an output layer of nodes. Connections between nodes may be created via the process of “training” the network, in which elements from a training data 504 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 5, machine-learning algorithms may include at least a supervised machine-learning process 528. At least a supervised machine-learning process 528, as defined herein, include algorithms that receive a training set relating a number of inputs to a number of outputs, and seek to generate one or more data structures representing and/or instantiating one or more mathematical relations relating inputs to outputs, where each of the one or more mathematical relations is optimal according to some criterion specified to the algorithm using some scoring function. For instance, a supervised learning algorithm may include the cooking style and the personal recipe as described above as inputs, recipe depiction as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 504. Persons skilled in the art, upon reviewing the entirety of this disclosure, will be aware of various possible variations of at least a supervised machine-learning process 528 that may be used to determine relation between inputs and outputs. Supervised machine-learning processes may include classification algorithms as defined above.

With further reference to FIG. 5, training a supervised machine-learning process may include, without limitation, iteratively updating coefficients, biases, weights based on an error function, expected loss, and/or risk function. For instance, an output generated by a supervised machine-learning model using an input example in a training example may be compared to an output example from the training example; an error function may be generated based on the comparison, which may include any error function suitable for use with any machine-learning algorithm described in this disclosure, including a square of a difference between one or more sets of compared values or the like. Such an error function may be used in turn to update one or more weights, biases, coefficients, or other parameters of a machine-learning model through any suitable process including without limitation gradient descent processes, least-squares processes, and/or other processes described in this disclosure. This may be done iteratively and/or recursively to gradually tune such weights, biases, coefficients, or other parameters. Updating may be performed, in neural networks, using one or more back-propagation algorithms. Iterative and/or recursive updates to weights, biases, coefficients, or other parameters as described above may be performed until currently available training data is exhausted and/or until a convergence test is passed, where a “convergence test” is a test for a condition selected as indicating that a model and/or weights, biases, coefficients, or other parameters thereof has reached a degree of accuracy. A convergence test may, for instance, compare a difference between two or more successive errors or error function values, where differences below a threshold amount may be taken to indicate convergence. Alternatively or additionally, one or more errors and/or error function values evaluated in training iterations may be compared to a threshold.

Still referring to FIG. 5, a computing device, processor, and/or module may be configured to perform method, method step, sequence of method steps and/or algorithm described in reference to this figure, in any order and with any degree of repetition. For instance, a computing device, processor, and/or module may be configured to perform a single step, sequence and/or algorithm repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. A computing device, processor, and/or module may perform any step, sequence of steps, or algorithm in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Further referring to FIG. 5, machine learning processes may include at least an unsupervised machine-learning processes 532. An unsupervised machine-learning process, as used herein, is a process that derives inferences in datasets without regard to labels; as a result, an unsupervised machine-learning process may be free to discover any structure, relationship, and/or correlation provided in the data. Unsupervised processes 532 may not require a response variable; unsupervised processes 532 may be used to find interesting patterns and/or inferences between variables, to determine a degree of correlation between two or more variables, or the like.

Still referring to FIG. 5, machine-learning module 500 may be designed and configured to create a machine-learning model 524 using techniques for development of linear regression models. Linear regression models may include ordinary least squares regression, which aims to minimize the square of the difference between predicted outcomes and actual outcomes according to an appropriate norm for measuring such a difference (e.g., a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 5, machine-learning algorithms may include, without limitation, linear discriminant analysis. Machine-learning algorithm may include quadratic discriminant analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized trees, AdaBoost, gradient tree boosting, and/or voting classifier methods. Machine-learning algorithms may include neural net algorithms, including convolutional neural net processes.

Still referring to FIG. 5, a machine-learning model and/or process may be deployed or instantiated by incorporation into a program, apparatus, system and/or module. For instance, and without limitation, a machine-learning model, neural network, and/or some or all parameters thereof may be stored and/or deployed in any memory or circuitry. Parameters such as coefficients, weights, and/or biases may be stored as circuit-based constants, such as arrays of wires and/or binary inputs and/or outputs set at logic “1” and “0” voltage levels in a logic circuit to represent a number according to any suitable encoding system including twos complement or the like or may be stored in any volatile and/or non-volatile memory. Similarly, mathematical operations and input and/or output of data to or from models, neural network layers, or the like may be instantiated in hardware circuitry and/or in the form of instructions in firmware, machine-code such as binary operation code instructions, assembly language, or any higher-order programming language. Any technology for hardware and/or software instantiation of memory, instructions, data structures, and/or algorithms may be used to instantiate a machine-learning process and/or model, including without limitation any combination of production and/or configuration of non-reconfigurable hardware elements, circuits, and/or modules such as without limitation ASICs, production and/or configuration of reconfigurable hardware elements, circuits, and/or modules such as without limitation FPGAs, production and/or of non-reconfigurable and/or configuration non-rewritable memory elements, circuits, and/or modules such as without limitation non-rewritable ROM, production and/or configuration of reconfigurable and/or rewritable memory elements, circuits, and/or modules such as without limitation rewritable ROM or other memory technology described in this disclosure, and/or production and/or configuration of any computing device and/or component thereof as described in this disclosure. Such deployed and/or instantiated machine-learning model and/or algorithm may receive inputs from any other process, module, and/or component described in this disclosure, and produce outputs to any other process, module, and/or component described in this disclosure.

Continuing to refer to FIG. 5, any process of training, retraining, deployment, and/or instantiation of any machine-learning model and/or algorithm may be performed and/or repeated after an initial deployment and/or instantiation to correct, refine, and/or improve the machine-learning model and/or algorithm. Such retraining, deployment, and/or instantiation may be performed as a periodic or regular process, such as retraining, deployment, and/or instantiation at regular elapsed time periods, after some measure of volume such as a number of bytes or other measures of data processed, a number of uses or performances of processes described in this disclosure, or the like, and/or according to a software, firmware, or other update schedule. Alternatively or additionally, retraining, deployment, and/or instantiation may be event-based, and may be triggered, without limitation, by user inputs indicating sub-optimal or otherwise problematic performance and/or by automated field testing and/or auditing processes, which may compare outputs of machine-learning models and/or algorithms, and/or errors and/or error functions thereof, to any thresholds, convergence tests, or the like, and/or may compare outputs of processes described herein to similar thresholds, convergence tests or the like. Event-based retraining, deployment, and/or instantiation may alternatively or additionally be triggered by receipt and/or generation of one or more new training examples; a number of new training examples may be compared to a preconfigured threshold, where exceeding the preconfigured threshold may trigger retraining, deployment, and/or instantiation.

Still referring to FIG. 5, retraining and/or additional training may be performed using any process for training described above, using any currently or previously deployed version of a machine-learning model and/or algorithm as a starting point. Training data for retraining may be collected, preconditioned, sorted, classified, sanitized, or otherwise processed according to any process described in this disclosure. Training data may include, without limitation, training examples including inputs and correlated outputs used, received, and/or generated from any version of any system, module, machine-learning model or algorithm, apparatus, and/or method described in this disclosure; such examples may be modified and/or labeled according to user feedback or other processes to indicate desired results, and/or may have actual or measured results from a process being modeled and/or predicted by system, module, machine-learning model or algorithm, apparatus, and/or method as “desired” results to be compared to outputs for training processes as described above.

Redeployment may be performed using any reconfiguring and/or rewriting of reconfigurable and/or rewritable circuit and/or memory elements; alternatively, redeployment may be performed by production of new hardware and/or software components, circuits, instructions, or the like, which may be added to and/or may replace existing hardware and/or software components, circuits, instructions, or the like.

Further referring to FIG. 5, one or more processes or algorithms described above may be performed by at least a dedicated hardware unit 536. A “dedicated hardware unit,” for the purposes of this figure, is a hardware component, circuit, or the like, aside from a principal control circuit and/or processor performing method steps as described in this disclosure, that is specifically designated or selected to perform one or more specific tasks and/or processes described in reference to this figure, such as without limitation preconditioning and/or sanitization of training data and/or training a machine-learning algorithm and/or model. A dedicated hardware unit 536 may include, without limitation, a hardware unit that can perform iterative or massed calculations, such as matrix-based calculations to update or tune parameters, weights, coefficients, and/or biases of machine-learning models and/or neural networks, efficiently using pipelining, parallel processing, or the like; such a hardware unit may be optimized for such processes by, for instance, including dedicated circuitry for matrix and/or signal processing operations that includes, e.g., multiple arithmetic and/or logical circuit units such as multipliers and/or adders that can act simultaneously and/or in parallel or the like. Such dedicated hardware units 536 may include, without limitation, graphical processing units (GPUs), dedicated signal processing modules, FPGA or other reconfigurable hardware that has been configured to instantiate parallel processing units for one or more specific tasks, or the like, A computing device, processor, apparatus, or module may be configured to instruct one or more dedicated hardware units 536 to perform one or more operations described herein, such as evaluation of model and/or algorithm outputs, one-time or iterative updates to parameters, coefficients, weights, and/or biases, and/or any other operations such as vector and/or matrix operations as described in this disclosure.

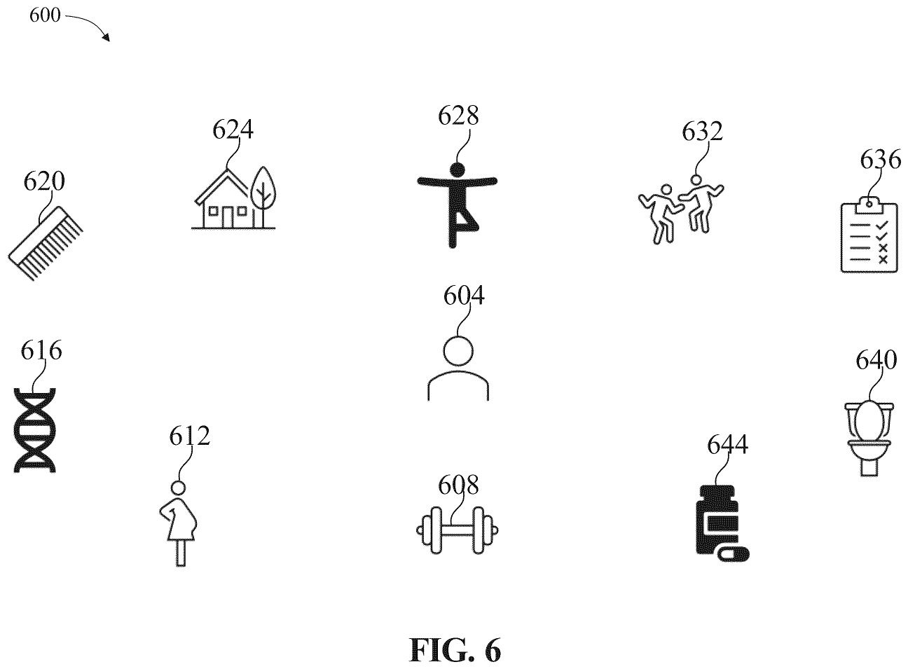

Referring now to FIG. 6, an exemplary embodiment 600 of information contained within user database 112 is illustrated. One or more entries contained within user database 112 may be utilized to generate and/or calculate target profile 108. User database 112 may contain information pertaining to a user 604, such as a human being. Entries contained within user database 112 may include fitness entries 608 which may describe information any fitness habits of a user. For example, a user may indicate that the user lifts weights on Tuesday and Thursday mornings at 6:00 am. Entries contained within user database 112 may include life stage information 612 which may describe a particular life stage that a user is currently at. For example, a female user may indicate if the user is currently pregnant which may cause the user to have extra nutritional demands and needs. Entries contained within user database 112 may include genetic entries 616, which may include any genetic tests and/or genetic results pertaining to a user. For example, a user may have a breast cancer gene mutation such as a BRCA1 or BRCA2 mutation, which may be detected based on previous genetic testing and/or sequencing. Entries contained within user database 112 may include hygiene entries 620, which may include any hygiene habits and/or products that the user frequently engages in. For example, a user may specify that the user purchases and uses non-toxic shampoo and conditioner but utilizes conventional toothpaste and facewash. Entries contained within user database 112 may include balance entries 628. Balance entries 628 may include information describing how flexible a user is, how often a user practices and/or engages in balance exercise, and types of coordinated balance exercise the user may engage in. Entries contained within user database 112 may include relaxation entries 632. Relaxation entries 632 may include information describing what sorts of activities a user engages in during time off from work and/or school, such as dancing, meditation, recreational activities, group exercise, hobbies, and the like. Entries contained within user database 112 may include questionnaire entries 636. Questionnaire entries 636 may include information describing a user's response to one or more questionnaires about the user's lifestyle and/or habits. Entries contained within user database 112 may include elimination entries 640. For example, elimination entries 640 may include a description of a user's eliminations and/or any gastrointestinal problems the user may experience, such as diarrhea, constipation, gas, bloating, and the like. Entries contained within user database 112 may include medication entries 644. Medication entries 644 may include information describing any prescription medications, herbal remedies, supplements, homeopathic remedies and the like that the user may consume.

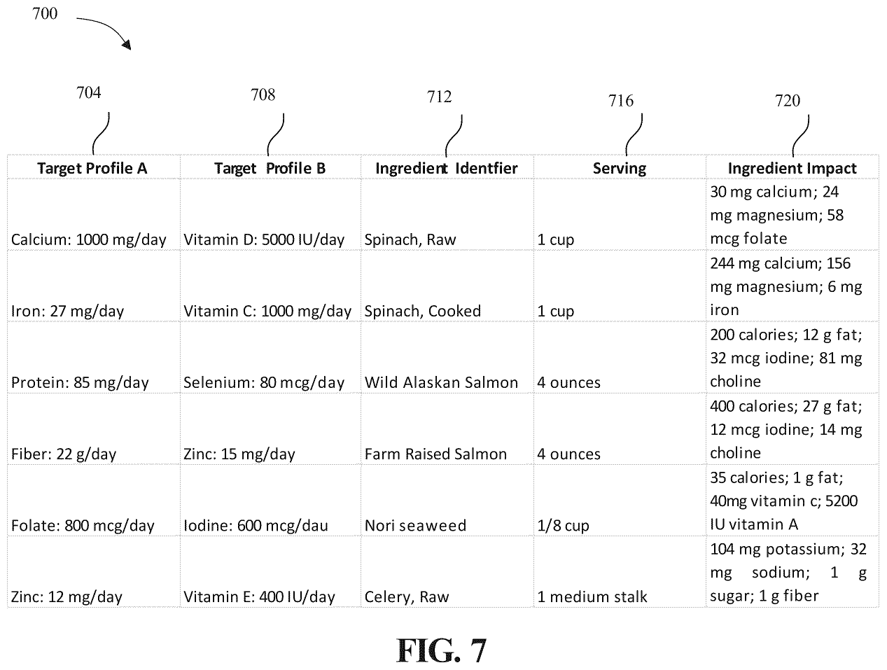

Referring now to FIG. 7, an exemplary embodiment 700 of target profiles and ingredient identifiers is illustrated. In an embodiment, target profile A 704 may be prepared and calculated for a user. In such an instance, target profile A 704 may contain one or more recommended nutrient intakes for a first user. For instance, and without limitation, target profile A 704 may recommend a user to consume 100 mg/day of calcium. In an embodiment, target profile A 704 may contain one or more recommended nutrient intakes for a specified period of time, such as the recommended intake per meal, per day, per week, and the like. In an embodiment, a period of time to report a recommended intake of a nutrient may be specified for the period of time as requested by a user, and/or a user preference contained within user database 112. In an embodiment, computing device 104 may calculate target profile B 708, which may contain one or more recommended nutrient intakes for a second user. In an embodiment, target profile A 704 may contain various nutrients and/or recommended doses which may be different from those contained within target profile B 708. For instance and without limitation, target profile A 704 may be generated for a first user who may be pregnant, and may require additional supplementation with nutrients such as calcium, iron, folate, and fiber whereas target profile B 708 may be generated for a second user who may not be pregnant and who may suffer from a medical condition such as hypothyroidism and may require recommended doses of nutrients such as selenium, zinc, and iodine. In an embodiment, an ingredient identifier 712 may specify the state of an ingredient, such as if an ingredient containing spinach is raw or cooked spinach or if fish includes wild caught fish or farmed fish. In an embodiment, a serving size 716 of an ingredient may be specified. In an embodiment, an ingredient impact 720 may contain the nutritional impact of one or more ingredients contained within an ingredient. For instance, and without limitation, the ingredient impact of raw spinach may specify that 1 cup of raw spinach may contain 30 mg calcium, 24 mg magnesium, and 58 mcg of folate. In an embodiment, computing device 104 may display nutrients contained within an ingredient impact that are most relevant and/or that match nutrients contained within a target profile. For example, a target profile that recommends a user to consume 15 mg of zinc per day may be utilized to locate the ingredient impact of an ingredient that contains zinc, whereas ingredients that do not contain zinc may not be selected and/or zinc may not be listed or contained within an ingredient impact for that particular ingredient. Information pertaining to serving sizes and/or ingredient impacts may be updated in real time utilizing any network methodology as described herein.

Referring now to FIG. 8, an exemplary embodiment of a filtering system is illustrated. Filtering system 800 may be implemented by computing device 104. As used in the current disclosure, a “filtering system” is a mechanism designed to refine the selection of ingredients from a comprehensive database of consumables down to a specific set that meets stringent criteria. This system may be used to ensure that only the most suitable and safe ingredients are chosen for any given recipe, adhering closely to user preferences and health guidelines.

With continued reference to FIG. 8, the first stage 804 of filtering system 800 may begin with a broad database of all consumables. This initial filtering may be comprehensive, covering a wide range of ingredients from common staples to exotic items. The purpose here may be to create a baseline from which more specific filtering criteria can be applied. This stage may involve removing items that do not meet basic safety standards or those that have been flagged for potential health risks.

With continued reference to FIG. 8, the second stage 808 of filtering system 800 may be focused on removing non-approved consumables, leaving only those that are approved for use. This stage may act as a critical filter to ensure that the ingredients list aligns with specific safety standards, dietary guidelines, or user-specified preferences and restrictions. In this stage, computing device 104 may employ a comprehensive set of criteria to evaluate each consumable. These criteria are derived from a variety of sources including regulatory safety standards, dietary guidelines from health organizations, and custom preferences set by the user, such as avoiding ingredients due to allergies, ethical reasons, or personal health concerns.

With continued reference to FIG. 8, the second stage 808 of the filtering system 800 may include several criteria that can be applied to ensure the safety and suitability of consumables. In an embodiment, the criteria may include an evaluation for regulatory compliance, ensuring they do not contain banned pesticides, harmful contaminants, and have not been implicated in recent food recalls. In an additional embodiment, the system 800 may examine whether the ingredients align with established dietary guidelines, such as those recommending low sodium for heart health or high fiber for better digestion. Additionally, user-preferences may play a crucial role in this stage; the system 800 may actively filter out ingredients that conflict with personal dietary preferences, such as allergens like nuts, religious dietary restrictions like pork, or any animal products for those following a vegan diet.

With continued reference to FIG. 8, the third stage 812 of the filtering process may target the removal of the “dirty dozen,” a term used to describe fruits and vegetables that are most contaminated with pesticides. This stage is crucial for minimizing the exposure to harmful chemicals. The filtering system identifies these items based on regularly updated environmental research and excludes them from the list of viable ingredients. Following the exclusion of the dirty dozen, the remaining ingredients may be filtered to ensure they are certified organic. This stage may be dedicated to promoting health and environmental benefits, focusing on ingredients that have been grown without the use of synthetic pesticides, genetically modified organisms (GMOs), and other practices that could compromise food quality and safety.

With continued reference to FIG. 8, after securing a list of organic ingredients, the filtering system 800 may apply a price filter. This may be used in making the selection process economically viable for the user. The price filter removes any organic ingredients that exceed a pre-set price threshold, which can be adjusted according to the user's budgetary constraints. This ensures that the final selection of ingredients aligns with both health standards and economic practicality.

With continued reference to FIG. 8, the fourth stage 816 of the filtering system 800 may target the ingredients that have passed through the previous filters, computing device 104 selects a group of ingredients that are not only safe and economically feasible but also suitable for the user's personal recipe 132. The selection of the user's personal recipe 132 from the lexicon of ingredients may be done in the fashion described in FIG. 1.

With continued reference to FIG. 8, the fifth stage 820 of the filtering system 800 may involve evaluating the ingredient accessibility of the selected ingredients. This crucial phase checks the real-time stock levels at local stores or suppliers, ensuring that each ingredient can indeed be purchased. This step prevents the frustration of planning a meal around ingredients that are currently unavailable, thereby streamlining the cooking process and ensuring user satisfaction. Evaluating the ingredient accessibility of the selected ingredients may be done in the fashion described above with reference with FIGS. 1-7.

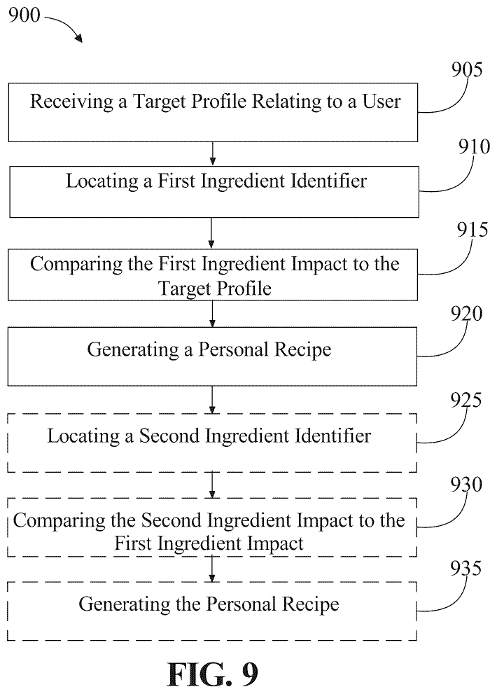

Referring now to FIG. 9, an exemplary embodiment 900 of a method of personal recipe generation is illustrated. At step 905, computing device 104 receives a target profile 108 relating to a user. Target profile 108 includes any of the target profiles 108 as described above in more detail in reference to FIG. 1. Target profile 108 may include one or more recommended nutrients for a user, such as for example due to a nutrient deficiency an. For instance, and without limitation, a target profile 108 may recommend that a user needs to consume 100 mg of thiamine per day and that the user should not consume more than 2000 mg of sodium per day. In yet another non-limiting example, target profile 108 may include a recommendation for a user to consume 35 grams of fiber, 400 mg of magnesium, and no more than 10 grams of sugar per day. Target profile 108 may identify a nutrient target computed for a measured time interval as described above in more detail. Target profile 108 may be determined using a nutrient anomaly 120. A nutrient anomaly 120 includes any of the nutrient anomalies as described above in more detail in reference to FIG. 1. A nutrient anomaly 120 may indicate an abnormality relating to a user's current nutrient status and/or requirements and may be utilized to generate a target profile 108. For instance and without limitation, a nutrient anomaly 120 may indicate that a user has high levels of b vitamins in the user's body, and as such, the user should consume no more than 500 mg per day of b vitamins. In yet another non-limiting example, a nutrient anomaly 120 may indicate that a user has very low levels of ferritin, and as such a target profile 108 may recommend for the user to consume 100 mg of ferrous sulfate daily in combination with 250 mg of vitamin c per day. Information relating to a nutrient anomaly 120 and/or information utilized to generate a target profile 108 may be stored within user database 112. In an embodiment, a nutrient anomaly 120 may contain a temporal element, including any of the temporal elements as described above in more detail in reference to FIG. 1. In an embodiment, a temporal element may specify how long a nutrient anomaly 120 has been ongoing. In an embodiment, a first nutrient anomaly 120 may be identified as a function of a second nutrient anomaly 120. For instance, and without limitation, a first nutrient anomaly 120 such as a deficiency of copper may be related to a second nutrient anomaly 120 such as an excess of zinc. In yet another non-limiting example, a first nutrient anomaly 120 such as an excess of omega 6 fatty acids may be related to a second nutrient anomaly 120 such as a deficiency of omega 9 fatty acids.

With continued reference to FIG. 9, at step 910 computing device 104 locates in a lexicon of ingredients a first ingredient identifier 124 including a first ingredient impact 132. First ingredient identifier 124 includes any of the first ingredient identifiers 124 as described above in more detail in reference to FIG. 1. First ingredient identifier 124 may identify a first ingredient. Information pertaining to first ingredient identifier 124 and/or ingredients may be contained within ingredient database 128. First ingredient identifier 124 includes a first ingredient impact 132. First ingredient impact 132 specifies nutrients contained within an ingredient. Computing device 104 locates a first ingredient identifier 124 based on information pertaining to a user's preference, which may be contained within user database 112. For instance, and without limitation, a user's preference to consume all animal proteins except for chicken may be utilized to select a first ingredient identifier 124 such as for a filet mignon steak. In yet another non-limiting example, a user's preference to consume a vegetarian diet may be utilized to select a first ingredient identifier 124 such as eggplant. Information pertaining to various ingredients may be stored and contained within ingredient database 128.

With continued reference to FIG. 9, at step 915 computing device 104 compares first ingredient impact 132 to target profile 108. Comparing may include determining how well first ingredient impact 132 meets and/or exceeds the nutritional target contained within target profile 108. For instance and without limitation, computing device 104 may compare first ingredient impact 132 for an ingredient such as cooked spinach, which contains 260 mcg of folate per cup, to a target profile 108 that recommends 200 mcg of folate per day. In an embodiment, computing device 104 may disregard a first ingredient identifier 124 that does not meet requirements contained within a target profile 108. For instance and without limitation, a first ingredient identifier 124 that specifies an ingredient such as a kiwi may not be selected to be included in personal recipe 136 if a target profile 108 specifies that a user should consume no more than 125 mg of Vitamin C per day, and a first ingredient impact 132 for the kiwi indicates that the kiwi contains 65 mg of Vitamin C.

With continued reference to FIG. 9, at step 920, computing device 104 generates a personal recipe 136. Personal recipe 136 includes any of the personal recipes as described above in more detail in reference to FIG. 1. At step 925, computing device 104 may generate personal recipe 136 by locating a second ingredient identifier 144, containing second ingredient impact 148. Second ingredient identifier 144 may be selected using information contained within recipe database 140. For instance and without limitation, recipe database 140 may contain an entry that a first ingredient identifier 124 such as fresh strawberries may be best matched with a second ingredient identifier 144 such as blueberry, blackberry, raspberry, basil, mint, cinnamon, vanilla, and/or cardamom. In such an instance, computing device 104 may select second ingredient identifier 144 from such an entry contained within recipe database 140. Computing device 104 may locate second ingredient identifier 144 by selecting a second ingredient that improves first ingredient impact 132. For instance, and without limitation, computing device 104 may locate second ingredient identifier 144 for an ingredient that enhances absorption and bioavailability of a first ingredient, thereby improving first ingredient impact 132. For example, computing device 104 may locate a second ingredient identifier 144 for an ingredient such as avocado, which may enhance absorption of Vitamin K found in romaine lettuce when both are paired and consumed together. At step 830, computing device 104 compares second ingredient impact 148 to first ingredient impact 132 and target profile 108. Comparing may include determining how second ingredient impact 148 may impact and/or affect first ingredient impact 132. For instance, and without limitation, a second ingredient may not be selected such as when the second ingredient impact 148 may adversely affect a first ingredient impact 132. For example, a first ingredient such as black tea may be adversely affected when combined with a second ingredient such as cow's milk, because the cow's milk may inhibit absorption of phytochemicals contained within black tea, thus adversely affecting first ingredient impact 132. In yet another non-limiting example, a first ingredient such as yogurt may be adversely affected when combined with a second ingredient such as blueberries, because the blueberries may inhibit absorption of calcium contained within the yogurt. At step 930, computing device 104 generates personal recipe 136 using first ingredient identifier 124 and second ingredient identifier 144. Generating personal recipe 136 is performed as a function of first semantic unit and second semantic unit. Computing device 104 may generate personal recipe 136 using a machine learning process 152 as described above in more detail in reference to FIGS. 1-5. Generating personal recipe 136 may include selecting a cooking style, as described above in more detail in reference to FIG. 1. In an embodiment, computing device 104 may select a cooking style using target profile 108. For instance and without limitation, computing device 104 may select a cooking style such as sautéing based on a target profile 108 that contains a recommendation for a user to consume no more than 15 grams of saturated fat per day, as compared to other cooking styles which may create high levels of saturated fat, such as frying.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

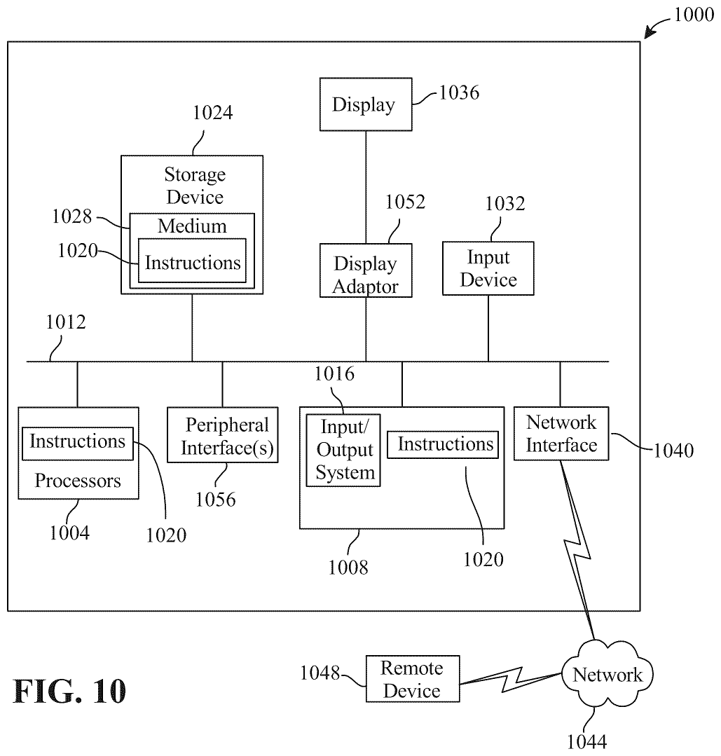

FIG. 10 shows a diagrammatic representation of one embodiment of a computing device in the exemplary form of a computer system 1000 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 1000 includes a processor 1004 and a memory 1008 that communicate with each other, and with other components, via a bus 1012. Bus 1012 may include any of several types of bus structures including, but not limited to, a memory bus, a memory controller, a peripheral bus, a local bus, and any combinations thereof, using any of a variety of bus architectures.

Processor 1004 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 1004 may be organized according to Von Neumann and/or Harvard architecture as a non-limiting example. Processor 1004 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC).

Memory 1008 may include various components (e.g., machine-readable media) including, but not limited to, a random-access memory component, a read only component, and any combinations thereof. In one example, a basic input/output system 1016 (BIOS), including basic routines that help to transfer information between elements within computer system 1000, such as during start-up, may be stored in memory 1008. Memory 1008 may also include (e.g., stored on one or more machine-readable media) instructions (e.g., software) 1020 embodying any one or more of the aspects and/or methodologies of the present disclosure. In another example, memory 1008 may further include any number of program modules including, but not limited to, an operating system, one or more application programs, other program modules, program data, and any combinations thereof.

Computer system 1000 may also include a storage device 1024. Examples of a storage device (e.g., storage device 1024) include, but are not limited to, a hard disk drive, a magnetic disk drive, an optical disc drive in combination with an optical medium, a solid-state memory device, and any combinations thereof. Storage device 1024 may be connected to bus 1012 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 1024 (or one or more components thereof) may be removably interfaced with computer system 1000 (e.g., via an external port connector (not shown)). Particularly, storage device 1024 and an associated machine-readable medium 1028 may provide nonvolatile and/or volatile storage of machine-readable instructions, data structures, program modules, and/or other data for computer system 1000. In one example, software 1020 may reside, completely or partially, within machine-readable medium 1028. In another example, software 1020 may reside, completely or partially, within processor 1004.

Computer system 1000 may also include an input device 1032. In one example, a user of computer system 1000 may enter commands and/or other information into computer system 1000 via input device 1032. Examples of an input device 1032 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 1032 may be interfaced to bus 1012 via any of a variety of interfaces (not shown) including, but not limited to, a serial interface, a parallel interface, a game port, a USB interface, a FIREWIRE interface, a direct interface to bus 1012, and any combinations thereof. Input device 1032 may include a touch screen interface that may be a part of or separate from display 1036, discussed further below. Input device 1032 may be utilized as a user selection device for selecting one or more graphical representations in a graphical interface as described above.

A user may also input commands and/or other information to computer system 1000 via storage device 1024 (e.g., a removable disk drive, a flash drive, etc.) and/or network interface device 1040. A network interface device, such as network interface device 1040, may be utilized for connecting computer system 1000 to one or more of a variety of networks, such as network 1044, and one or more remote devices 1048 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 1044, may employ a wired and/or a wireless mode of communication. In general, any network topology may be used. Information (e.g., data, software 1020, etc.) may be communicated to and/or from computer system 1000 via network interface device 1040.

Computer system 1000 may further include a video display adapter 1052 for communicating a displayable image to a display device, such as display device 1036. Examples of a display device include, but are not limited to, a liquid crystal display (LCD), a cathode ray tube (CRT), a plasma display, a light emitting diode (LED) display, and any combinations thereof. Display adapter 1052 and display device 1036 may be utilized in combination with processor 1004 to provide graphical representations of aspects of the present disclosure. In addition to a display device, computer system 1000 may include one or more other peripheral output devices including, but not limited to, an audio speaker, a printer, and any combinations thereof. Such peripheral output devices may be connected to bus 1012 via a peripheral interface 1056. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions, and additions may be made to that which is specifically disclosed herein without departing from the spirit and scope of the present invention.

What is claimed is:

1. A system for informing cooking style decisions, the system comprising a computing device, the computing device designed and configured to:
identify a nutrient anomaly relating to a user;
determine a target profile utilizing the nutrient anomaly;
generate a personal recipe relating to the user, wherein generating the personal recipe comprises:
selecting a plurality of ingredient identifiers as a function of the target profile, wherein each ingredient identifier is associated with an ingredient impact and an ingredient accessibility;
filtering the plurality of ingredient identifiers as a function of the ingredient accessibilities; and
generating the personal recipe using a plurality of ingredient impacts associated with the filtered plurality of ingredient identifiers;
select a cooking style, wherein selecting the cooking style comprises:
locating, in a recipe database, a cooking style table comprising a set of cooking styles associated with the personal recipe; and
selecting the cooking style from the set of cooking styles as a function of the target profile; and
generate a recipe depiction as a function of the cooking style and the personal recipe.

2. The system of claim 1, wherein the recipe depiction comprises an instructional video describing a preparation of the personal recipe in the selected cooking style.

3. The system of claim 1, wherein the computing device is further configured to classify the personal recipe and the cooking style to one or more meal categories.

4. The system of claim 1, wherein the computing device is further configured to classify the personal recipe and the cooking style to one or more cuisine categories.

5. The system of claim 1, wherein selecting the cooking style from the set of cooking styles comprises selecting the cooking style from the set of cooking styles as a function of a user preference.

6. The system of claim 1, wherein selecting the cooking style from the set of cooking styles comprises selecting the cooking style from the set of cooking styles as a function of a temporal component.

7. The system of claim 1, wherein selecting the plurality of ingredient identifiers comprises locating the plurality of ingredient identifiers within a lexicon of ingredients.

8. The system of claim 7, wherein the lexicon of ingredients comprises associations between ingredient identifiers, ingredient impacts, and semantic units for each ingredient identifier of the plurality of ingredient identifiers.

9. The system of claim 1, wherein filtering the plurality of ingredient identifiers comprises generating an accessibility score for each ingredient identifier of the plurality of ingredient identifiers.

10. The system of claim 9, wherein filtering the plurality of ingredient identifiers further comprises:
comparing, for each ingredient identifier of the plurality of ingredient identifiers, the accessibility scores to a threshold; and
filtering the plurality of ingredient identifiers as a function of the comparison.

11. A method for informing cooking style decisions, wherein the method comprises:
identifying, using at least a computing device, a nutrient anomaly relating to a user;
determining, using the at least a computing device, a target profile utilizing the nutrient anomaly;
generating, using the at least a computing device, a personal recipe relating to the user, wherein generating the personal recipe comprises:
selecting a plurality of ingredient identifiers as a function of the target profile, wherein each ingredient identifier is associated with an ingredient impact and an ingredient accessibility;
filtering the plurality of ingredient identifiers as a function of the ingredient accessibilities; and
generating the personal recipe using a plurality of ingredient impacts associated with the filtered plurality of ingredient identifiers;
selecting, using the at least a computing device, a cooking style, wherein selecting the cooking style comprises:
locating, in a recipe database, a cooking style table comprising a set of cooking styles associated with the personal recipe; and
selecting the cooking style from the set of cooking styles as a function of the target profile; and
generating, using the at least a computing device, a recipe depiction as a function of the cooking style and the personal recipe.

12. The method of claim 11, wherein the recipe depiction comprises an instructional video describing a preparation of the personal recipe in the selected cooking style.

13. The method of claim 11, wherein the method further comprises classifying, using the at least a computing device, the personal recipe and the cooking style to one or more meal categories.

14. The method of claim 11, wherein the method further comprises classifying, using the at least a computing device, the personal recipe and the cooking style to one or more cuisine categories.

15. The method of claim 11, wherein selecting the cooking style from the set of cooking styles comprises selecting the cooking style from the set of cooking styles as a function of a user preference.

16. The method of claim 11, wherein selecting the cooking style from the set of cooking styles comprises selecting the cooking style from the set of cooking styles as a function of a temporal component.

17. The method of claim 11, wherein selecting the plurality of ingredient identifiers comprises locating the plurality of ingredient identifiers within a lexicon of ingredients.

18. The method of claim 17, wherein the lexicon of ingredients comprises associations between ingredient identifiers, ingredient impacts, and a semantic units for each ingredient identifier of the plurality of ingredient identifiers.

19. The method of claim 11, wherein filtering the plurality of ingredient identifiers comprises generating an accessibility score for each ingredient identifier of the plurality of ingredient identifiers.

20. The method of claim 19, wherein filtering the plurality of ingredient identifiers further comprises:
comparing, for each ingredient identifier of the plurality of ingredient identifiers, the accessibility scores to a threshold; and
filtering the plurality of ingredient identifiers as a function of the comparison.
